(19) United States
(12) Patent Application Publication
(10) Pub. No.: US 2003/0068365 A1
(43) Pub. Date: Apr. 10, 2003

(54) Compositions and methods for administration of active agents using liposome beads

(76) Inventors: Pichit Suvanprakorn, Bangkok (TH); Tanusin Ploysangam, Bangkok (TH); Lerson Tanasugarn, Bangkok (TH); Suwalee Chandrkrachang, Bangkok (TH); Nardo Zaias, Miami Beach, FL (US)

Correspondence Address: Law Office of Eric G. Masamori, 6520 Ridgewood Drive, Castro Valley, CA, 94552, US

(21) Appl. No.: 10/264,205

(22) Filed: Oct. 3, 2002

Related U.S. Application Data
(60) Provisional application No. 60/327,643, filed on Oct. 5, 2001.

(51) Int. Cl. A61K 9/127; A61K 35/78

(52) U.S. Cl. 424/450; 424/725

(57) ABSTRACT

Compositions and methods for administration of active agents encapsulated within liposome beads to enable a wider range of delivery vehicles, to provide longer product shelf-life, to allow multiple active agents within the composition, to allow the controlled use of the active agents, to provide protected and designable release features and to provide visual inspection for damage and inconsistency.

### CROSS REFERENCE TO OTHER APPLICATIONS

This application claims the benefit of U.S. Provisional Patent Application No. 60/327,643 filed Oct. 5, 2001.

### FIELD OF INVENTION

The present invention relates to compositions and methods for administration of active agents, including but not limited to cosmetic, cosmeceuticals and pharmaceuticals, to biological organisms in need thereof. More specifically, the present invention relates to aggregated or globulized multiple encapsulations of active agents using conventionally prepared liposomes and aggregating or globulizing those liposomes into globules or beads.

### BACKGROUND OF THE INVENTION

When phospholipids and many other amphipathic lipids are dispersed gently in an aqueous medium they hydrate and spontaneously form multilamellar concentric bilayer vesicles. The lipid bilayers are separated with layers of the aqueous media. These vesicles are commonly referred to as multilamellar liposomes or multilamellar vesicles and usually have diameters of about 0.2 μm to 5 μm. Sonication of the multilamellar vesicles results in the formation of smaller unilamellar vesicles with diameters usually in the range of 20 to 100 nm, containing an aqueous solution in the core. Multivesicular liposomes differ from multilamellar liposomes in the random, non-concentric arrangement of the chambers within the liposome. Amphipathic lipids can form a variety of structures other than liposomes when dispersed in water, depending on the molar ratio of lipid to water, but at low ratios the liposome is the preferred structure.

The physical characteristics of liposomes generally depend on pH and ionic strength. They characteristically show low permeability to ionic and polar substances, but at certain temperatures can undergo a gel-liquid crystalline phase transition dependent upon the physical properties of the lipids used in their manufacture which markedly alters their permeability. The phase transition involves a change from a closely packed, ordered structure, known as the gel state, to a loosely packed, less ordered structure, known as the liquid crystalline phase.

Various types of lipids differing in chain length, saturation, and head group have been used in liposomal drug formulations for many years, including the unilamellar, multilamellar and multivesicular liposomes described above. The major goal of the field is to develop liposomal formulations for sustained release of drugs and other compounds of interest and to develop liposome formulations from which the rate of release of the encapsulated material can be controlled.

Various limitations on the shelf-life and use of liposome compounds exist due to the relatively fragile nature of liposomes. Major problems encountered during liposome drug storage in vesicular suspension are the chemical alterations of the liposome compounds, such as phospholipids, cholesterols, ceramides, leading to potentially toxic degradation of the products, leakage of the drug from the liposome and the modifications of the size and morphology of the phospholipid liposome vesicles through aggregation and fusion. Liposome vesicles are known to be thermodynamically relatively unstable at room temperature and can spontaneously fuse into larger, less stable altered liposome forms.

Also adding to the potential instability of liposomes in conventional formulations is the pKa. The pKa of compounds may be defined by the pH at which concentrations of both the uncharged and charged forms of the molecules are found.

Various schemes have been devised to avoid some the stability and limitations of liposome formulations, such as freeze drying of the composition. The freeze dried composition is reconstituted as required for use.

What is needed is a liposome formulation that avoids the disadvantages of pre-existing liposomes formulations discussed above, that has a longer shelf-life, provides controlled and increased concentrations of active agents at or near the desired target administration site, allows segregation of different active agents and provides the ability to visually determine if the integrity of the liposome has been affected by undesired alterations.

### SUMMARY OF THE INVENTION

The present invention contemplates the use of liposome encapsulated materials made by any conventional means and subsequently, or in addition to the encapsulation process, provides a system to suspend these liposomes into discrete multilamellar vesicles or beads. The multilamellar vesicles or beads are designed with surface tensions of different strengths to provide an improved delivery system of a drug or other active agent. The present invention provides compositions and methods of administration of globules or beads of liposomal formulations and active agents in predetermined sizes with similar or different active agents, thereby enhancing the use of the drug or active agents in a number of different ways.

Accordingly, the present invention provides a composition and method of administration of active agents which, when used in combination with liposomes, enables a wider range of vehicles, provides longer life of the product, provides controlled and increased concentrations of active agents at or near the desired target administration site, provides protected and designable release features, allows segregation of different active agents and allows the controlled use of the active agent and their visual inspection for damage and consistency.

In general, the invention comprises a composition and method for the administration of beads or globules of liposomal formulations and active agents. The active agents include but are not limited to cosmetics, cosmeceuticals and pharmaceuticals. A liposomal suspension of multilamellar vesicles encapsulating the active agent is prepared by conventional methods. The liposomal suspension is placed into a physical or physiochemical bonding solution resulting in a liposomal first solution. The resulting liposomal first solution is then aliquoted into a second solution containing at least one inorganic salt. The at least one inorganic salt of the second solution comprises 1-2% by weight of the second solution. Upon entry into the second solution, the liposomal first solution develops a hardened surface and forms a bead. The beads are then aggregated and washed with an inert solution to remove any residual liposomal first solution and second solution. The resulting liposomal beads are now ready for use.

In the preferred embodiment, multiple portions of an empty aqueous liposome formulation are lyophilized and hydrated with a solution of active agent or other material that are to be encapsulated resulting in the formation of liposome multilamellar vesicles containing the active agent or materials. In the preferred embodiment, the active agent is selected from the group consisting of cosmetics, cosmeceuticals and pharmaceuticals. However, in alternate embodiment, it may be possible for one skilled in the art to use other materials with different therapeutic characteristics. The portions of liposome solution are then separated or pooled to form the final liposome preparation. Each batch may be washed prior to pooling to remove unencapsulated active agent. In the preferred liposome encapsulation process, 50 to 95% of the total active agent or other material is entrapped or encapsulated. Alternative methods of preparing the liposome preparation may be used, as will be readily apparent to one skilled in the art.

The liposome multilamellar vesicles are then mixed into a vessel containing a pre-determined concentration of a physical and/or potentially physiochemical bonding solution. This mixture results in a liposomal first solution. In the preferred embodiment, the bonding solution contains at least one organic compound selected from the group consisting of aragrose, cellulose, sodium alginate, chitosans, or polymeric substances. In alternate embodiments, other compounds with the necessary characteristics of physical or physiochemical bonding may be used.

In another preferred embodiment, the liposomal first solution is comprised of the multilamellar liposome containing cosmetic or pharmaceutical actives mixed with a micro-emulsion solution composed of organic oils in one phase and a group of organic compounds consisting of aragrose, cellulose, sodium alginate, chitosans, or polymeric substances at a pre-determined temperature.

The preferred bonding characteristics include the ability to form polymer network attraction, compatible with liposomes, able to form beads in the presence of inorganic salts. The bonding may consist of polarity bonding, ionic bonding, Van der Waals bonding and affinity bonding.

It is preferable that the bonding solution forms the outer shell of the bead in the presence of inorganic salts and holds the liposomal actives inside at the same time to maintain the stability of the bead and enhance the stability of the liposomes. The bonding solution can also protect the inner microparticle liposomes when exposed to the inorganic salts. The general concentration range of the bonding solution depends upon the type of the bead desired; however the preferred concentration range is 1 to 1.5% by weight. Different beads require different concentrations of the bonding solution to provide the proper degree of hardness of the shell.

The liposomal first solution is then introduced into a second solution comprising one or more inorganic salts. In the preferred embodiment, the organic salt is selected from the group consisting of calcium chloride, calcium sulfate, calcium carbonate, mangesium chloride, magnesiun sulfate, barium chloride, or barium sulfate. In alternate embodiments, other inorganic salts may be used in the second solution. In the preferred embodiment, the inorganic salt comprises about 1 to 2% by weight of the second solution.

In the preferred embodiment, the liposomal first solution is introduced into the inorganic salt solution through a predetermined orifice which allows for a specific size or amount of liposomal first solution to be introduced. In prototype development testing, the types of delivery systems used included needle injection and disc spinning. However, other types of delivery systems, such as spraying, hydraulic pressure pump, gravitational dipping, pneumatic pumping or liquidating methods may be used. The means of bead formation can be achieved by a number of alternative embodiments, including but not limited to providing the liposome formulation through a spray, spinning vessels, injection, pumping, dripping or aliquoting method.

Upon entry of the liposomal first solution into the inorganic salt solution, the liposomal first solution develops a hardened outer surface and forms a bead. In the preferred embodiment, the beads are generally spherical or irregular polygon in shape and their appearance allows for identification and verification of bead formation. The shape, degree of hardening and resulting force necessary to fracture the bead is determined by the formulation of the inorganic salt solution, the pH of the inorganic sal solution, the time of submersion or contact with the inorganic salt solution, and the relative temperature differentials between the liposome formulation and the inorganic salt solution.

In the preferred embodiment, the pH of the inorganic salt solution was 6-7, the length of time of submersion was 30 to 45 minutes, and the solution temperature was 25 to 30° C.

The hardness of the bead is measured in “yield strength”, which is measured as the amount of weight required to rupture the bead. The yield strength is expressed as grams per cubic millimeter (gm/mm³). The preferred range of hardness or force necessary to fracture the bead is 1 to 4 gm/mm³. However, the range of firmness may vary, so long as the liposome formulation remains constituted in bead form.

The bead are physically separated by any means of selection, specific gravity or physical filtration and rinsed with any conventional washing operation. In the preferred embodiment, the beads are separated by a sieve and washed with deionized water for 15 minutes and then rinsed again with deionized water. The outer portions of the wet liposome embodiments, including liposome-micro emulsion spheres, are then dehydrated to remove the remaining water. Dehydration is accomplished by any chemical and/or physical means. The dried liposome micro-emulsion spheres are then stored in a pre-determined concentration of organic, inorganic, or aqueous aliquot of organic or inorganic compound solution, ready to be further processed into finished products.

The liposome beads can be used in any number of delivery vehicles. The variability and uses of the beaded liposome are extensive with the physical characteristics and applications being determined and designed by the physical characteristics of the bead wall and the contents of the bead.

In one embodiment, carbopol gel will be used for oil-soluble actives. The carbopol gels may be neutralized by means of alkaline substances or buffered by a predetermined pH buffer solution to yield clear gels.

In another embodiment, silicone derivatives will be used for water soluble actives. The silicone derivatives vehicles are designed such that an anhydrous environment is achieved and the clarity and/or viscosity are adjusted through the quantities of the organic silicones or solvents comprising the silicone bases of intended use.

The now prepared final bead formulation can be used for any of the desired embodiments. The variability and uses of the beaded liposome are extensive with the physical characteristics and applications being determined and designed by the physical characteristics of the bead wall and the contents of the bead.

The benefits of defined beads of liposome include one or more of the following for each use:

The therapeutic benefit of treating all types of Dermatocytosis.

The therapeutic benefit from the user being able to provide controlled and increased concentration of active agent released at or near the desired target site of administration on or in the skin.

The therapeutic benefit of allowing the user to visually determine the location and amount of the active agent applied to the treatment area thus enabling the user to control the locus and levels of agent where the active ingredient is most needed.

The benefit of having active agents in bead form, and thus not in direct contact with other active or inert suspensions including other liposomes. This includes second and third levels of bead formation and levels of hardening encapsulation.

The benefit of being able to produce liposome beads of discrete and predetermined size for more accurate administration of drugs or other active agents.

The benefit of having different types of delivery vehicles, containing the beads, to deliver the treatment, whether inert or containing active agents, thereby effectively allowing for the vehicle to be an active agent.

The benefit of having more than one active agent encapsulated within the bead membrane itself, thereby providing a multiple active agent liposomal mixture which only becomes interactive when the dynamics of the beads are affected to release their encapsulated agents.

The benefit of being able to define the physical characteristics of the semi-rigid wall of the bead for specific protection and delivery of the liposomic compound or compounds, including a slow and/or continuous delivery of the active agent.

The benefit of a bead protecting the liposomal formulation from physically changing forces such as ultrasound, vibration, light, microwaves and other energy providing sources, by both the semi-rigid wall and the type of vehicles used.

The benefit of designing extended shelf life and protection of the liposome through various means including the suspension of the beads in vehicles, which would ordinarily adversely react with a liposome.

The ability to visually determine if any undesired alterations or lack of integrity bead wall has affected the integrity of the liposome is also a benefit. A broken or distended bead can indicate the potential instability of the liposomes.

The benefit of a hardened surface wall which can then be processed in various means for special uses by means and compounds that would otherwise have been damaging or altering to the liposomal active agents.

The benefit of easily designing and producing a variety of different bead walls and differently controlled releases of active agents, released through a number of different mechanisms for maintaining or delivering a liposome at the desired site of the delivery, which delivery can be released and controlled by the fracture of the bead wall through various means of wall release. Thus, the entire design becomes a tool for the effective delivery individually or as part of a system which requires the addition of outside energy or intervention.

The benefit of a designed indicator of any liposome degradation, infiltration or loss of bead wall integrity.

### DETAILED DESCRIPTION OF THE INVENTION

The present invention contemplates the use of liposome encapsulated materials made by any conventional means and subsequently, or in addition to the encapsulation process, provides a composition to suspend these liposomes into discrete multilamellar vesicles or beads. The multilamellar vesicles or beads are designed with surface tensions of different strengths to provide an improved delivery system of a drug or other active agent. The present invention is a composition and method of administration of globules or beads of liposomal formulations and active agents in predetermined sizes with similar or different active agents, thereby enhancing the use of the drug or active agents in a number of different ways.

The present invention provides a composition and method of administration which, when added to any other active or inactive delivery of liposomes, enables a wider range of vehicles, provides longer life of the product, allows additional active agents within the compound, provides protected and designable release features, and allows the controlled use of the active agent and their visual inspection for damage and consistency. The active agents being selected from the group consisting of cosmetics, cosmeceuticals and pharmaceuticals.

The present invention is compatible with all known and anticipated liposomal structures and results in predetermined sizes of globulized beads allowing for a second and additional level of control, shelf life and application ease. Liposome compositions, which have this additional step of placing the liposome into beads, have been shown to be more effective in the delivery of the active agent in several means. They also enjoy superior or increased shelf life of the active agent, and allow different active agents to remain segregated until release upon fracture of the bead surface. They also allow for the storage of normally incompatible active agents in one composition to be delivered to the biological organism.

In the preferred embodiment, the intended liposome is made by any known means of formation. Typical liposome manufacturing processes comprise the following steps: multiple portions of an “empty” aqueous liposome formulation are provided, each portion is lyophilized and hydrated with a solution of the active agents or materials that are to be encapsulated, resulting in the formation of liposomes which have trapped the active agent or material. These portions are then separated or pooled to form a batch of material, which typically constitutes the final liposome preparation. Each batch may be washed prior to pooling to remove unencapsulated material.

Alternatively, liposomes are prepared using an organic solution of lipids which are dried and hydrated with water to form “empty” liposome formulations. Each portion is then lyophilized and hydrated with a solution of the material to be encapsulated.

In another alternative procedure, liposome formulation compounds are made by lyophilizing an empty liposome formulation and aliquoting the lyophilized material into a plurality of portions prior to lyophilization. Each lyophilized portion is then hydrated with a solution of the material that is to be encapsulated, and may be washed to remove unencapsulated material.

In a fourth alternative procedure, a plurality of portions of an organic solution of lipids is provided in a plurality of containers, and the organic solvents are evaporated from each portion, resulting in the formation of a thin lipid film on the walls of each container. The evaporation process may be any conventional evaporation process, such as rotary evaporation. An aqueous solution of the material to be encapsulated is then added to each portion and the container is agitated. The resulting solution is the formation of a plurality of portions of liposomes that have trapped the material. These portions are then pooled to form the final liposome preparation.

In an adaptation of the fourth alternative process described above, an aqueous solution of a material to be encapsulated is added to one the plurality of containers which have the thin lipid film on the walls, and this container is agitated to hydrate the lipid film and form a liposome suspension. This suspension is then added to another container having the thin lipid film on the walls thereof. This container is agitated to hydrate the lipid film. This process is repeated until all of the containers having the thin lipid film have been hydrated, resulting in the formation of the final liposome preparation.

Other conventional approaches to making liposome mixtures may be used, such as rotating systems to encapsulate the active form in a suspension or the use of an aqueous solution, which is under pressure, and is injected with the active agent into a lipid solution to form liposomes, referred to as “reverse phasing method”.

It is preferable that the selected liposome encapsulation process traps or encapsulates 50 to 95% of the available total active agents. It is preferable that the active agent comprise 0.01 to 5 weight percent of the liposome composition.

The prepared liposome is then mixed into a vessel containing a predetermined concentration of a physical and potentially physiochemical bonding solution. It is preferable that the bonding solution contains at least one organic compound such as aragrose, cellulose, sodium alginate, chitosans, polymeric substances or other compounds with the necessary characteristic of physical or physiochemical bonding. The resulting solution is hereinafter referred to as the “liposomal first solution”.

In another preferred embodiment, the liposomal first solution comprises the multilamellar liposome containing cosmetic or pharmaceutical actives mixed with a micro-emulsion solution composed of organic oils in one phase and a group of organic compounds consisting of aragrose, cellulose, sodium alginate, chitosans, or polymeric substances at a pre-determined temperature.

The preferred physical or physiochemical bonding characteristics include the ability to form polymer network attraction, compatible with liposomes, able to form beads in the presence of inorganic salts. The bonding may consist of polarity bonding, ionic bonding, Van der Waals bonding and affinity bonding.

It is preferable that the bonding solution forms the outer shell of the bead in the presence of inorganic salts and holds the liposomal actives inside at the same time to maintain the stability of the bead and enhance the stability of the liposomes. The solution bonding can also protect the inner microparticle liposomes when exposed to the inorganic salts. The general concentration range of the bonding solution depends upon the type of the bead; however the preferred concentration range is 1 to 1.5% by wieght. Different beads require different concentrations of the bonding solution to provide the proper degree of hardness of the shell.

The liposomal first solution is preferably introduced into a second solution comprising one or more inorganic salts through a predetermined orifice which allows for a specific size or amount of liposomal first solution one to be introduced into the second solution. The inorganic salt preferably comprises about 1 to 2% by weight of the second solution. The effect of the interaction of the liposomal first solution with the second solution is to harden the outer most exposed areas of the introduced liposomal first solution. In prototype testing the second solution was comprised of calcium chloride or sodium hydroxide, although other types of inorganic salts can be used such as calcium sulfate, calcium carbonate, magnesium chloride, magnesium sulfate, barium chloride, barium sulfate or other salts.

In the preferred embodiment, the liposomal first solution is introduced into the inorganic salt solution by dripping the liposomal first solution through a small needle or predetermined orifice or by spinning the liposomal first solution with a centrifugal force via a rotating disc. The predetermined orifice allows for a specific size or amount of liposome solution to be introduced. In prototype development testing, other types of delivery system also used included spraying, hydraulic pressure pump, gravitational dipping, pneumatic pumping or liquidating methods.

In another preferred embodiment, the liposomal first solution comprises the multilamellar liposome containing cosmetic or pharmaceutical activce mixed with a micro-emulsion solution composed of organic oils in one phase and a group of organic compounds consisting of aragrose, cellulose, sodium alginate, chitosans or polymeric substances. The liposome-micro-emulsion solution is then introduced into the inorganic salt solution through a predetermined orifice which allows for a specific size or amount of lipsome micro-emulsion solution to be introduced.

Upon entry into the second solution, the liposomal first solution develops a hardened surface and forms a bead, typically 1 to 4 mm in size. Differing appearances allow for identification and verification of the formation and size of the bead. The shape, degree of hardening and resulting force necessary to fracture the bead in order to release its active ingredient is determined by the formulation of the second solution, the pH of the second solution, the time of submersion or contact with the second solution, and the relative temperature differentials.

The preferred general shape of the formed bead is generally spherical or irregular polygon. The hardness of the bead is measured in “yield strength”, which is measured as the amount of weight required to rupture the bead. The yield strength is expressed as grams per cubic millimeter (gm/mm³). The preferred range of hardness or force necessary to fracture the bead is 1 to 4 gm/mm³. However, the range of firmness may vary, so long as the liposome formulation remains constituted in bead form.

The beads are physically separated by any means of selection, specific gravity or physical filtration and rinsed with any conventional washing operation. In the preferred embodiment, the beads are separated by a sieve and washed with deionized water for 15 minutes and then rinsed again with deionized water. The outer portions of the wet liposome embodiments, including liposome-micro emulsion spheres, are then dehydrated to remove the remaining water. The dehydration process is accomplished by any chemical and/or physical means. The dried liposome micro-emulsion spheres are then stored in a pre-determined concentration of organic, inorganic, or aqueous aliquot of organic or inorganic compound solution, ready to be further processed into finished products.

Depending upon the designed use of the liposome bead, various compositions are achieved by changes to the surface thickness of the bead, the size of the bead, the shape of the bead, and any additional compounds which are added to the delivery vehicle.

The now prepared final bead composition can be used in a multitude of applications. The variability and uses of the beaded liposome are extensive with the physical characteristics and applications being determined and designed by the physical characteristics of the bead wall and the contents of the bead.

In one preferred embodiment the liposome encapsulated bead composition is used for topical application. The liposome encapsulated bead composition comprises a therapeutically effective amount of an active agent encapsulated in a liposome suspension of multilamellar vesicles in an amount from about 0.01 to 5 weight percent based on the weight of the whole composition, in admixture with a physical bonding solution wherein the admixture is subsequently encapsulated within at least one inorganic salt.

In another preferred embodiment, the invention relates to a composition and method of administering:

(a) a therapeutically effective amount of a first active agent encapsulated in a liposome suspension of multilamellar vesicles

(b) a therapeutically effective amount of a second active agent encapsulated in a liposome suspension of multilamellar vesicles

(c) in admixture with the liposome suspension of multilamellar vesicles a physical bonding solution

wherein each active agents comprises 0.01 to 5% by weight of the total composition and wherein the admixture is subsequently encapsulated within at least one inorganic salt.

In another embodiment, the invention relates to a composition and method of administering one or more active agents to a subject comprising the steps of:

(a) providing a liposome encapsulated bead composition containing at least one active agent, and

(b) contacting an area of skin or mucous membrane of a subject with the composition to administer the at least one active agent

The term active agent as used in the specification sections entitled “Summary of the Invention” and “Detailed Description of the Invention” and in the above examples is intended to include the following therapeutic categories: topically applied antifungals, such as Terbinafine, Ketoconazole, Climbazole, Tolnaftate; anti-inflammatories (nonsteroidal); antiarthritics; corticosteroids, such as Clobetasone, Triancinolone acetonide, Betamethasone; vitamins, such as Retinoic Acid, Vitamin K1, Nicotinamide, Vitamin E; whitening agents, such as Arbutin, AHAs; antioxidants, such as Tranexamic Acid, Polyphenols; nitrus oxide, moisturizers, such as Aloe vera and Evening primrose oil; anabolics; analgesics (dental, narcotic and non-narcotic); anesthetics (local); antiasthmatics (nonbronchodilator, steroidal, inhalant); antibacterial (antibiotics); antihistaminics; antineoplastics; antiparasitics; vasodilators; vasoconstrictors, anti-tumor, i.e., seborrheic keratosis to malignant tumors such as basal cell carcinoma; anti-viral (warts and molluscum contagiosum); anti seborrheic; anti-vertigo such as compazine; anti insects (anti lice); deliverance of toxins, such as botox (nerve paralysis); deliverance of hormones such as estrogen; delivery of nicotine; prophylactic uses of many of the above; for anti cold; for release of heat; and prevention of contact dermatitis and irritants.

The term active agent is also intended to include the following categories:

Vitamins, such as: Vitamin A/Beta-Carotene, Vitamin B1 (Thiamin), Vitamin B3 (Niacin), Vitamin B6, Vitamin B12, Biotin, Folic Acid, Pantothenic Acid and Pantethine, Vitamin C, Vitamin D, Vitamin E, Vitamin K

Minerals, such as: Boron, Calcium, Chromium, Copper, Fluorine, Germanium, Iodine, Iron, Magnesium, Manganese, Molybdenum, Phosphorus, Potassium, Selenium, Silicon, Vanadium, Zinc

Amino Acids, such as: L-Arginine, L-Aspartic Acid, Branched-Chain Amino Acids, L-Cysteine (and Glutathione), L-Glutamine/L-Glutamic Acid, Glycine, L-Histidine, L-Lysine, L-Methionine and Taurine, L-Phenylalanine, D-Phenylalanine, DL-Phenylalanine, L-Tryptophan, L-Tyrosine

Lipids, such as: AL, Fish Oils/EPA and DHA, Gamma-Linolenic Acid and Oil of Evening Primrose, Glycosphingolipids, Inositol (Myo-Inositol) and Phosphatidylinositol, Lecithin/Phosphatidylcholine/Choline, Liposomes, Lipotropes/Activated Lipotropes, Monolaurin and Caprylic Acid, Phosphatidylserine and Phosphatidylethanolamine

Herbs, such as: Aconite, Alfalfa, Aloe Vera and Derivatives, Angelica/Dong Quai, Astragalus, Bayberry Root Bark, Black Cohosh, Blessed Thistle, Buchu, Burdock, Butcher's Broom, Capsicum/Hot Peppers, Cascara Sagrada, Catnip, Chamomile, Chaparral, Chickweed, Comfrey/Allantoin, Cruciferous Vegetables, Damiana, Dandelion, Devil's Claw, Echinacea, Ephedra/Ma-Huang, Euphorbia, Eyebright, Fennel, Fenugreek, Feverfew, Forskolin, Fo-Ti, Garlic and Onions, Ginger, Ginkgo, Ginseng, Goldenseal, Gotu Kola, Hawthorn, Herbal Analbesic Ointments and Oils, Herbal Fiber, Horsetail Grass, Juniper, Kava Kava, Licorice, Ligustrum, Melaleuca, Marshmallow, Mexican Wild Yam, Milk Thistle, Mistletoe/Iscador, Mullein, Myrrh, Nettle, Oats, Parsley, Pau d'arco, Quinine, Red Clover, Red Raspberry, Saint John's Wort, Sarsaparilla, Schizandra, Senna, Skullcap, Slippery Elm, Triphala, Uva Ursi, Valerian, Walnuts, Wheat Grass/Barley Grass, White Oak, Yellow Dock, Yohimbine

Metabolite Supplements, such as: Acidophilus/Yogurt/Kefir, Bioflavonoids, Brewer's Yeast/Skin Respiratory Factor/Glucan, Coenzyme Q, Dietary Fiber, Enzymes, L-Carnitine, Lipoic Acid, Mushrooms: Shiitake and Rei-Shi, PABA, Panagamic Acid/DMG (“Vitamin B-15”), Royal Jelly, Seaweeds and Derivatives, Spirulina and Chlorella, Succinates and Cytochromes, Wheat Germ/Wheat-Germ Oil/Octacosanol.

The term “administering” is intended to mean any mode of application to a tissue, which results in the physical contact of the composition with an anatomical site. The term “subject” is intended to include all biological organisms.

In accordance with one embodiment, the liposome beads are introduced into an inert delivery vehicle or solution, such as lotions, ointments, creams or sprays, for its use.

Another embodiment provides for the liposome beads to be contained in an inert delivery solution which is translucent or opaque to the desired level of light reduction.

Another embodiment provides for the liposome beads to be fractured by a mechanical means as the delivery solution is metered or dispensed from a device. The preferred fracturing means can be an orifice which is significantly smaller than the size of the particular bead, however any known fracturing means may be utilized.

Another embodiment provides for the liposome bead walls to be degraded by nonphysical chemical means, including both pre-existing chemical conditions or the introduction of a degrading chemical through other means such as within the delivery vehicle, or with other liposome beads.

Another embodiment provides for the liposome beads to be coated with a particular color or pattern of recognition so as to allow the user to meter and judge the amount of active reagent without unnecessary dilution.

Another embodiment provides for the size of the liposome beads to change in response to any changes to the liposome occurring within the bead wall, thereby indicating a potentially compromised liposome bead.

Another embodiment provides for the liposome beads to be suspended in chronologically degrading walls, or bead walls that are altered by enzymatic or pH factors, such as the enzymes and pH changes found when administered systemically. The liposome bead can be designed to allow the active agents to remain protected until fracture or surface tension release by the appropriate enzyme, chronological passage, or pH change.

Another embodiment provides for various degrees of hardening of the liposome bead wall, the degree of hardening being predetermined to provide for greater or lesser forces to cause the degradation of the bead wall and release of its contents at distinct intervals or levels.

Another embodiment provides for coating the liposome beads with various compounds, which are reactive to the active agent. The incompatible agents are separated by the hardened shell, and only become interactive upon the fracture or softening of the bead wall.

Anther embodiment provides for mechanical means of release on the using of apparel, for example shoes, to activate the fracture or destruction of the bead wall, so as to release the active agent.

Another embodiment provides for the use of a photoactive suspension of the vehicle so as to release the liposome from the bead on the event of a predetermined condition or level of light or other waveform, or any other energy transmission, such as ultrasound, microwave, light or percussion forces.

Another embodiment provides for the use of a chemically reactive vehicle compound which, upon the event of the vehicle coming into contact with its reactive counterpart, the liposome bead wall is fractured and the liposome released.

Another embodiment provides for the fracture through temperature sensitive bead walls, with relative temperatures providing relative release points.

The foregoing list of therapeutic categories and various embodiments are illustrative of the invention and are merely exemplary. A person skilled in the art may make variations and modifications without departing from the spirit and scope of the invention. All such modifications and variations are intended to be included within the scope of the invention as described in this specification.

Indeed, the present invention is intended to encompass and be suitable for use by substituting any of the following drugs for the active agent in the composition and methods for administration of the same:

alpha-ADRENERGIC AGONIST such as Adrafinil, Adrenolone, Amidephrine, Apraclonidine, Budralazine, Clonidine, Cyclopentamine, Detomidine, Dimetofrine, Dipivefrin, Ephedrine, Epinephrine, Fenoxazoline, Guanabenz, Guanfacine, Hydroxyamphetamine, Ibopamine, Indanazoline, Isometheptene, Mephentermine, Metaraminol, Methoxamine, Methylhexaneamine, Metizolene, Midodrine, Modafinil, Moxonidine, Naphazoline, Norepinephrine, Norfenefrine, Octodrine, Octopamine, Oxymetazoline, Phenylephrine, Phenylpropanolamine, Phenylpropylmethylamine, Pholedrine, Propylhexedrine, Pseudoephedrine, Rilmenidine, Synephrine, Talipexole, Tetrahydrozoline, Tiamenidine, Tramazoline, Tuaminoheptane, Tymazoline, Tyramine, Xylometazoline

beta-ADRENERGIC AGONIST such as Albuterol, Bambuterol, Bitolterol, Carbuterol, Clenbuterol, Clorprenaline, Denopamine, Ephedrine, Epinephrine, Etafedrine, Ethylnorepinephrine, Fenoterol, Formoterol, Hexoprenaline, Ibopamine, Isoetharine, Isoproterenol, Mabuterol, Metaproterenol, Methoxyphenamine, Oxyfedrine, Pirbuterol, Prenalterol, Procaterol, Protokylol, Reproterol, Rimiterol, Ritodrine, Salmerterol, Soterenol, Terbutaline, Tretoquinol, Tulobuterol, Xamoterol

alpha-ADRENERGIC BLOCKER such as Amosulalol, Arotinilol, Dapiprazole, Doxazosin, Ergoloid Mesylates, Fenspiride, Indoramine, Labetalol, Naftopidil, Nicergoline, Prazosin, Tamsulosin, Terazosin, Tolazoline, Trimazosin, Yohimbine

beta-ADRENERGIC BLOCKER such as Acebutolol, Alprenolol, Amosulalol, Arotinolol, Atenolol, Befunolol, Betaxolol, Bevantolol, Bisoprolol, Bopindolol, Bucumolol, Bufetolol, Bufuralol, Bunitrolol, Bupranolol, Butidrine, Butofilolol, Carazolol, Carteolol, Carvedilol, Celiprolol, Cetamolol, Cloranolol, Dilevalol, Epanolol, Esmolol, Indenolol, Labetalol, Levobunolol, Mepindolol, Metipranolol, Metoprolol, Moprolol, Nadolol, Nadoxolol, Nebivalol, Nifenalol, Nipradilol, Oxprenolol, Penbutolol, Pindolol, Practolol, Pronethalol, Propranolol, Sotalol, Sulfinalol, Talinolol, Tertatolol, Tilisolol, Timolol, Toliprolol, Xibenolol

ALCOHOL DETERRENT such as Calcium Cyanamide Citrated, Disulfiram, Nitrefazole

ALDOSE REDUCTASE INHIBITOR such as Epalrestat, Sorbinil, Tolrestat, Zopolrestat

ANABOLIC such as Androisoxazole, Androstenediol, Bolandiol, Bolasterone, Clostebol, Ethylestrenol, Formebolone, Methandriol, Methenolone, Methyltrienolone, Nandrolone, Norbolethone, Oxabolone, Oxymesterone, Pizotyline, Quinbolone, Stenbolone, Trenbolone

ANALGESIC (DENTAL) such as Chlorobutanol, Clove, Eugenol

ANALGESIC (NARCOTIC) such as Alfentanil, Allyiprodine, Alphaprodine, Anileridine, Benzylmorphine, Bezitramide, Buprenorphine, Butorphanol, Clonitazene, Codeines, Desomorphine, Dextromoramide, Dezocine, Diampromide, Dihydrocodeine, Dihydrocodeinone Enol Acetate, Dihydromorphine, Dimenoxadol, Dimepheptanol, Dimethylthiambutene, Dioxaphetyl Butyrate, Dipipanone, Eptazocine, Ethoheptazine, Ethylmethlythiambutene, Ethylmorphine, Etonitazene, Fentanyl, Hydrocodone, Hydromorphone, Hydroxypethidine, Isomethadone, Ketobemidone, Levorphanol, Lofentanil, Meperidine, Meptazinol, Metazocine, Methadone, Metopon, Morphine, Morphine Derivatives, Myrophine, Nalbuphine, Narceine, Nicomorphine, Norlevorphanol, Normethadone, Normorphine, Norpipanone, Opium, Oxycodone, Oxymorphone, Papaveretum, Pentazocine, Phenadoxone, Phenazocine, Phenoperidine, Piminodine, Piritramide, Proheptazine, Promedol, Propiram, Propoxyphene, Remefentanil, Sufentanil, Tilidine

ANALGESIC (NON-NARCOTIC) such as Aceclofenac, Acetaminophen, Acetaminosalol, Acetanilide, Acetylsalicylsalicylic Acid, Alclofenac, Alminoprofen, Aloxiprin, Aluminum Bis(acetylsalicylate), Aminochlorthenoxazin, 2-Amino-4-picoline, Aminopropylon, Aminopyrine, Ammonium Salicylate, Amtolmetin Guacil, Antipyrine, Antipyrine Salicylate, Antrafenine, Apazone, Aspirin, Benorylate, Benoxaprofen, Benzpiperylon, Benzydamine, Bermoprofen, Bromofenac, p-Bromoacetanilide, 5-Bromosalicylic Acid Acetate, Bucetin, Bufexamac, Bumadizon, Butacetin, Calcium Acetylsalicylate, Carbamazepine, Carbiphene, Carsalam, Chloralantipyrine, Chlorthenoxazin(e), Choline Salicylate, Cinchophen, Ciramadol, Clometacin, Clonixin, Cropropamide, Crotethamide, Dexoxadrol, Difenamizole, Diflunisal, Dihydroxyaluminum Acetylsalicylate, Dipyrocetyl, Dipyrone, Emorfazone, Enfenamic Acid, Epirizole, Etersalate, Ethenzamide, Ethoxazene, Etodolac, Felbinac, Fenoprofen, Floctafenine, Flufenamic Acid, Fluoresone, Flupirtine, Fluproquazone, Flurbiprofen, Fosfosal, Gentisic Acid, Glafenine, Ibufenac, Imidazole Salicylate, Indomethacin, Indoprofen, Isofezolac, Isoladol, Isonixin, Ketoprofen, Ketorolac, p-Lactophenetide, Lefetamine, Lornoxicam, Loxoprofen, Lysine Acetylsalicylate, Magnesium Acetylsalicylate, Methotrimeprazine, Metofoline, Mofezolac, Morazone, Morpholine Salicylate, Naproxen, Nefopam, Nifenazone, 5′ Nitro-2′ propoxyacetanilide, Parsalmide, Perisoxal, Phenacetin, Phenazopyridine, Phenocoll, Phenopyrazone, Phenyl Acetylsalicylate, Phenyl Salicylate, Phenyramidol, Pipebuzone, Piperylone, Propacetamol, Propyphenazone, Ramifenazone, Rimazolium Metilsulfate Salacetamide, Salicin, Salicylamide, Salicylamide O-Acetic Acid, Salicylsulfuric Acid, Salsalate, Salverine, Simetride, Sodium Salicylate, Suprofen, Talniflumate, Tenoxicam, Terofenamate, Tetradrine, Tinoridine, Tolfenamic Acid, Tramadol, Tropesin, Viminol, Xenbucin, Zomepirac

ANDROGEN such as Boldenone, Cloxotestosterone, Fluoxymesterone, Mestanolone, Mesterolone, Methandrostenolone, 17-Methyltestosterone, 17 alpha-Methyl-testosterone 3-Cyclopentyl Enol Ether, Norethandrolone, Normethandrone, Oxandrolone, Oxymesterone, Oxymetholone, Prasterone, Stanlolone, Stanozolol, Testosterone, Tiomesterone

ANESTHETIC such as Acetamidoeugenol, Alfadolone Acetate, Alfaxalone, Ambucaine, Amolanone, Amylocaine, Benoxinate, Benzocaine, Betoxycaine, Biphenamine, Bupivacaine, Butacaine, Butamben, Butanilicaine, Butethamine, Buthalital, Butoxycaine, Carticaine, Chloroprocaine, Cocaethylene, Cocaine, Cyclomethycaine, Dibucaine, Dimethisoquin, Dimethocaine, Diperadon, Dyclonine, Ecgonidine, Ecgonine, Ethyl Chloride, Etidocaine, Etoxadrol, beta-Eucaine, Euprocin, Fenalcomine, Fomocaine, Hexobarbital, Hexylcaine, Hydroxydione, Hydroxyprocaine, Hydroxytetracaine, Isobutyl p-Aminobenzoate, Ketamine, Leucinocaine Mesylate, Levoxadrol, Lidocaine, Mepivacaine, Meprylcaine, Metabutoxycaine, Methohexital, Methyl Chloride, Midazolam, Myrtecaine, Naepaine, Octacaine, Orthocaine, Oxethazaine, Parethoxycaine, Phenacaine, Phencyclidine, Phenol, Piperocaine, Piridocaine, Polidocanol, Pramoxine, Prilocaine, Procaine, Propanidid, Propanocaine, Proparacaine, Propipocaine, Propofol, Propoxycaine, Pseudococaine, Pyrrocaine, Ropivacaine, Salicyl Alcohol, Sodium Oxybate, Tetracaine, Thialbarbital, Thiamylal, Thiobutabarbital, Thiopental, Tolycaine, Trimecaine, Zolamine

ANOREXIC such as Aminorex, Amphecloral, Amphetamine, Benzaphetamine, Chlorphentermine, Clobenzorex, Cloforex, Clortermine, Cyclexedrine, Dextroamphetamine, Diethylpropion, Diphemethoxidine, N-Ethylamphetamine Fenbutrazate, Fenfluramine, Fenproporex, Furfurylmethylamphetamine, Levophacetoperate, Mazindol, Mefenorex, Metamfeproamone, Metamphetamine, Norpseudoephedrine, Pentorex, Phendimetrazine, Phenmetrazine, Phenpentermine, Phenylpropanolamine, Picilorex, Sibutramine ANTHELMINTIC (CESTODES) such as Arecoline, Aspidin, Aspidinol, Dichlorophen(e), Embelin, Kosin, Napthalene, Niclosamide, Pellertierine, Quinacrine

ANTHELMINTIC (NEMATODES) such as Alantolactone, Amocarzine, Amoscanate, Ascaridole, Bephenium, Bitoscanate, Carbon Tetrachloride, Carvacrol, Cyclobendazole, Diethylcarbamazine, Diphenane, Dithiazanine Iodide, Dymanthine, Gentian Violet, 4-Hexylresorcinol, Ivermectin, Kainic Acid, Levamisole, Mebendazole, 2-Napthol, Oxantel, Papain, Piperazines, Pyrantel, Pyrvinium Pamoate, alpha-Santonin, Stilbazium Iodide, Tetrachloroethylene, Thiabendazole, Thymol, Thymyl N-Isoamylcarbamate, Triclofenol Piperazine, Urea Stibamine

ANTHELMINTIC (SCHISTOSOMA) such as Amoscanate, Amphotalide, Antimony(s) and Derivatives, Becanthone, Hycanthone, Lucanthone, Niridazole, Oxamniquine, Praziquantel, Stibocaptate, Stibophen, Urea Stibamine

ANTHELMINTIC (TREMATODES) such as Anthiolimine, Tetrachloroethylene

ANTIACNE such as Algestone Acetophenide, Azelaic Acid, Benzoyl Peroxide, Cioteronel, Cyproterone, Motretinide, Resorcinol, Retinoic Acid, Tazarotene, Tetroquinone, Tioxolone

ANTIALLERGIC such as Amlexanox, Astemizole, Azelastine, Cromolyn, Fenpiprane, Ibudilast, Lodoxamide, Nedocromil, Oxatomide, Pemirolast, Pentigetide, Picumast, Repirinast, Suplast Tosylate, Tranilast, Traxanox

ANTIAMEBIC such as Arsthinol, Bialamicol, Carbarsone, Cephaeline, Chlorbetamide, Chloroquinone, Chlorphenoxamide, Chlorotetracycline, Dehydroemetine, Dibromopropamidine, Diloxanide, Dephetarsone, Emetine, Fumagillin, Glaucarubin, Glycobiarsol, 8-Hydroxy-7-iodo-5-quinolinesulfonic Acid, Iodochlorhydroxyquin, Iodoquinol, Paromomycin, Phanquinone, Polybenzarsol, Propamidine, Quinfamide, Secnidazole, Sulfarside, Teclozan, Tetracycline, Thiocarbamizine, Thiocarbarsone, Tinidazole

ANTIANDROGEN such as Bicalutamide, Bifluranol, Cioteronel, Cyproterone, Delmadinone Acetate, Flutamide, Nilutamide, Osaterone, Oxendolone

ANTIANGINAL such as Acebutolol, Alprenolol, Amiodarone, Amlodipine, Arotinolol, Atenolol, Barnidipine, Bepridil, Bevantolol, Bucumolol, Bufetolol, Bufuralol, Bunitrolol, Bupranolol, Carazolol, Carteolol, Celiprolol, Cinepazet Maleate, Diltazem, Elgodipine, Epanolol, Felodipine, Gallopamil, Imolamine, Indenolol, Isosorbide Dinitrate, Isradipine, Limaprost, Mepindolol, Metoprolol, Molsidomine, Nadolol, Nicardipine, Nicorandil, Nifedipine, Nifenalol, Nilvadipine, Nipradilol, Nitroglycerin,Oxprenolol, Oxyfedrine, Ozagrel, Penbutolol, Pentaerythritol Tetranitrate, Pindolol, Pronethalol, Propranolol, Ranolazine, Semotiadil, Sotalol, Terodiline, Timolol, Toliprolol, Trolnitrate Phosphate, Verapamil, Zatebradine

ANTIARRHYTHMIC such as Acebutolol, Acecainide, Adenosine, Ajmaline, Alprenolol, Amiodarone, Amoproxan, Aprindine, Arotinolol, Atenolol, Azimilide, Bevantolol, Bidisomide, Bretylium Tosylate, Bucumolol, Bufetolol, Bunaftine, Bunitrolol, Bupranolol, Butidrine, Butobendine, Capobenic Acid, Carazolol, Carteolol, Cifenline, Disopyramide, Dofetilide, Encainide, Esmolol, Flecainide, Hydroquinidine, Ibutilide, Indecainide, Indenolol, Ipratropium, Lidocaine, Lorajmine, Lorcainide, Meobentine, Mexiletine, Moricizine, Nadoxolol, Nifenalol, Oxprenolol, Penbutolol, Pilsicainide, Pindolol, Pirmenol, Practolol, Prajmaline, Procainamide, Pronethalol, Propafenone, Propranolol, Pyrinoline, Quinidine, Sematilide, Sotalol, Talinolol, Tilisolol, Timolol, Tocainide, Verapamil,, Viquidil, Xibenolol

ANTIARTERIOSCLEROTIC such as Pyridinol Carbamate

ANTIARTHRITIC/ANTIRHEUMATIC such as Actarit, Allocupreide Sodium, Auranofin, Aurothioglucose, Aurothioglycanide, Azathioprine, Bucillamine, Calcium 3-Aurothio-2propanol-1-sulfonate, Cloroquine, Clobuzarit, Cuproxoline, Diacerein, Glucosamine, Gold Sodium Thiomalate, Gold Sodium Thiosulfate, Hydroxychloroquine, Kebuzone, Lobenzarit, Melittin, Methotrexate, Myoral, Penicillamine

Antiarteriosclerotic (Antibiotic)

Aminoglycosides such as Amikacin, Apramycin, Arbekacin, Bambermycins, Butirosin, Dibekacin, Dihdrostreptomycin, Fortimicin(s), Fradiomycin, Gentamicin, Ispamicin, Kanamycin, Micronomicin, Neomycin, Neomycin Undecylenate, Netilmicin, Paromomycin, Ribostamycin, Sisomicin, Spectinomycin, Streptomycin, Tobramycin, Trospectomycin

Amphenicols such as Azidamfenicol, Chloramphenicol, Florfenicol, Thiamphenicol

Ansamycins such as Rifamide, Rifampin, Rifamycin, Rifapentine, Rifaximin beta-Lactams

Carbapenems such as Biapenem, Imipenem, Meropenem, Panipenem

Cephalosporins such as Cefaclor, Cefadroxil, Cefamandole, Cefatrizine, Cefazedone, Cefazolin, Cefcapene Pivoxil, Cefclidin, Cefdinir, Cefditoren, Cefepime, Cefetamet, Cefixime, Cefmenoxime, Cefodizime, Cefonicid, Cefoperazone, Ceforanide, Cefotaxime, Cefotiam, Cefozopran, Cefpimizole, Cefpiramide, Cefpirome, Cefpodoxime Proxetil, Cefprozil, Cefroxadine, Cefsulodin, Ceftazidime, Cefteram, Ceftezole, Ceftibuten, Ceftizoxime, Ceftriaxone, Cefuroxime, Cefuzonam, Cephacetrile Sodium, Cephalexin, Cephaloglycin, Cephaloridine, Cephalosporin, Cephalothin, Cephapirin Sodium, Cephradine, Pivcefalexin

Cephamycins such as Cefbuperazone, Cefmetazole, Cefminox, Cefetan, Cefoxitin

Monobactams such as Aztreonam, Carumonam, Tigemonam

Oxacephems such as Flomoxef, Moxolactam

Penicillins such as Amidinocillin, Amdinocillin Pivoxil, Amoxicillin, Ampicillan, Apalcillin, Aspoxicillin, Azidocillan, Azlocillan, Bacampicillin, Benzylpenicillinic Acid, Benzylpenicillin, Carbenicillin,, Carindacillin, Clometocillin, Cloxacillin, Cyclacillin, Dicloxacillin, Epicillin, Fenbenicillin, Floxicillin, Hetacillin, Lenampicillin, Metampicillin, Methicillin, Mezlocillin, Nafcillin, Oxacillin, Penamecillin, Penethamate Hydriodide, Penicillin G Benethamine, Penicillin G Benzathine, Penicillin G Benzhydrylamine, Penicillin G Calcium, Penicillin G Hydrabamine, Penicillin G Potassium, Penicillin G Procaine, Penicillin N, Penicillin O, Penicillin V, Penicillin V Benzathine, Penicillin V Hydrabamine, Penimepicycline, Phenethicillin, Piperacillin, Pivapicillin, Propicillin, Quinacillin, Sulbenicillin, Sultamicillin, Talampicillin, Temocillin, Ticarcillin

Others such as Ritipenem Lincosamides such as Clindamycin, Lincomycin

Macrolides such as Azithromycin, Carbomycin, Clarithromycin, Dirithromycin, Erythromycin(s) and Derivatives, Josamycin, Leucomycins, Midecamycins, Miokamycin, Oleandomycin, Primycin, Rokitamycin, Rosaramicin, Roxithromycin, Spiramycin, Troleandomycin

Polypeptides such as Amphomycin, Bacitracin, Capreomycin, Colistin, Enduracidin, Enviomycin, Fusafungine, Gramicidin(s), Gramicidin S, Mikamycin, Polymyxin, Pristinamycin, Ristocetin, Teicoplanin, Thiostrepton, Tuberactinomycin, Tyrocidine, Tyrothricin, Vancomycin, Viomycin(s), Virginiamycin, Zinc Bacitracin

Tetracyclines such as Apicycline, Chlortetracycline, Clomocycline, Demeclocycline, Doxycycline, Guamecycline, Lymecycline, Meclocycline, Methacycline, Minocycline, Oxytetracycline, Penimepicycline, Pipacycline, Rolitetracycline, Sancycline, Tetracycline

Others such as Cycloserine, Mupirocin, Tuberin,

Antibacterial (Synthetic)

2,4-Diaminopyrimidines such as Brodimoprim, Tetroxoprim, Trimethoprim

Nitrofurans such as Furaltadone, Furazolium, Nifuradene, Nifuratel, Nifurfoline, Nifurpirinol, Nifurprazine, Nifurtoinol, Nitrofurantoin

Quinolones and Analogs such as Cinoxacin, Ciprofloxacin, Clinafloxacin, Difloxacin, Enoxacin, Fleroxacin, Flumequine, Grepafloxacin, Lomefloxacin, Miloxacin, Nalidixic Acid, Norfloxacin, Ofloxacin, Oxolinic Acid, Pazufloxacin, Pefloxacin, Pipemidic Acid, Piromidic Acid, Rosoxacin, Rufloxacin, Sparfloxacin, Temafloxacin, Tosufloxacin, Travafloxacin

Sulfonamides such as Acetyl Sulfamethoxypyrazine, Benzylsulfamide, Chloramine-B, Chloramine-T, Dichloramine T, N²-Formyl-sulfisomidine, N⁴-beta.-D-Glucosylsulfanilamide, Mafenide, 4′-(Methyl-sulfamoyl)sulfanilanilide, Noprylsulfamide, Phthalylsulfacetamide, Phthalylsulfathiazole, Salazosulfadimidine, Succinylsulfathiazole, Sulfabenzamide, Sulfacetamide, Sulfachlorpyridazine, Sulfachrysoidine, Sulfacytine, Sulfadiazine, Sulfadicramide, Sulfadimethoxine, Sulfadoxine, Sulfaethidole, Sulfaguanidine, Sulfaguanol, Sulfalene, Sulfaloxic Acid, Sulfamerazine, Sulfameter, Sulfamethazine, Sulfamethizole, Sulfamethomidine, Sulfamethoxazole, Sulfamethoxypyridazine, Sulfametrole, Sulfamidochrysoidine, Sulfamoxole, Sulfanilamide, 4-Sulfanilamidosalicylic Acid, N⁴-Sulfanilylsulfanilamide, Sulfanilylurea, N-Sulfanilyl-3,4-xylamide, Sulfanitran, Sulfaperine, Sulfaphenazole, Sulfaproxyline, Sulfapyrazine, Sulfapyridine, Sulfasomizole, Sulfasymazine, Sulfathiazole, Sulfathiourea, Sulfatolamide, Sulfisomidine, Sulfisoxazole

Sulfones such as Acedapsone, Acediasulfone, Acetosulfone, Dapsone, Diathymosulfone, Glucosulfone, Solasulfone, Succisulfone, Sulfanilic Acid, p-Sulfanilylbenzylamine, Sulfoxone, Thiazolsulfone

Others such as Clofoctol, Hexedine, Methenamine, Methenamine Anhydromethylene-citrate, Methenamine Hippurate, Methenamine Mandelate, Methenamine Sulfosalicylate, Nitroxoline, Taurolidine, Xibornol

ANTICHOLINERGIC such as Adiphenine, Alverine, Ambutonomium, Aminopentamide, Amixetrine, Amprotropine Phosphate, Anisotropine Methylbromide, Apoatropine, Atropine, Atropine N-Oxide, Benactyzine, Benapryzine, Benzetimide, Benzilonium, Benztropine Mesylate, Bevonium Methyl Sulfate, Biperiden, Butropium, N-Butylscopolammonium Bromide, Buzepide, Camylofine, Caramiphen, Chlorbenzoxamine, Chlorphenoxamine, Cimetropium, Clidinium, Cyclodrine, Cyclonium, Cycrimine, Deptropine, Dexetimide, Dibutoline Sulfate, Dicyclomine, Diethazine, Difemerine, Dihexyverine, Diphemanil Methylsulfate, N-(1,2-Diphenylethyl)nicotinamide, Dipiproverine, Diponium, Emepronium, Endobenzyline, Ethopropazine, Ethybenztropine, Ethylbenzhydramine, Etomidoline, Eucatropine, Fenpiverinium, Fentonium, Flutropium, Glycopyrrolate, Heteronium, Hexocyclium Methyl Sulfate, Homatropine, Hyoscyamine, Ipratropium, Isopropamide, Levomepate, Mecloxamine, Mepenzolate, Metcaraphen, Methantheline, Methixene, Methscopolamine, Octamylamine, Oxybutynin, Oxyphencyclimine, Oxyphenonium, Pentapiperide, Penthienate, Phencarbamide, Phenglutarimide, Pipenzolate, Piperidolate, Piperilate, Poldine Methysulfate, Pridinol, Prifinium, Procyclidine, Propantheline, Propenzolate, Propiverine, Propyromazine, Scopolamine, Scopolamine N-Oxide, Stramonium, Sultroponium, Thiphenamil, Tiemonium, Timepidium, Tiquizium, Tridihexethyl Iodide, Trihexyphenidyl Hydrochloride, Trimebutine, Tropacine, Tropenzile, Tropicamide, Trospium, Valethamate, Vamicamide, Xenytropium

ANTICONVULSANT such as Acetylpheneturide, Albutoin, Aloxidone, Aminoglutethimide, 4-Amino-3-hydroxybutyric Acid, Atrolactamide, Beclamide, Buramate, Calcium Bromide, Carbamazepine, Cinromide, Clomethiazole, Clonazepam, Decimemide, Diethadione, Dimethadione, Doxenitoin, Eterobarb, Ethadione, Ethosuximide, Ethotoin, Felbamate, Fluoresone, Gabapentin, 5-Hydroxytryptophan, Lamotrigine, Magnesium Bromide, Magnesium Sulfate, Mephenytoin, Methobarbital, Metharbital, Methetoin, Methsuximide, 5-Methyl-5-(3-phenanthryl)-hydantoin, 3-Methyl-5-phenylhydantoin, Narcobarbital, Nimetazepam, Nitrazepam, Oxcarbazepine, Paramethadione, Phenacemide, Phenetharbital, Pheneturide, Phenobarbital, Phensuximide, Phenylmethylbarbituric Acid, Phenytoin, Phethenylate Sodium, Potassium Bromide, Primidone, Progabide, Sodium Bromide, Solanum, Strontium, Suclofenide, Sulthiame, Tetrantoin, Tiagabine, Topiramate, Trimethadione, Valproic Acid, Valpromide, Vigabatrin, Zonisamide

Antidepressant

Bicyclics such as Binedaline, Caroxazone, Citalopram, Dimethazan, Indalpine, Fencamine, Indeloxazine, Nefopam, Nomifensine, Oxitriptan, Oxypertine, Paroxetine, Sertraline, Thiazesim, Trazodone

Hydrazides/Hydrazines such as Benmoxine, Iproclozide, Iproniazid, Isocarboxazid, Nialamide, Octamoxin, Phenelzine

Pyrrolidones such as Cotinine, Rolicyprine, Rolipram

Tetracyclics such as Maprotiline, Metralindole, Mianserin, Oxaprotiline

Tricyclics such as Adinazolam, Amitriptyline, Amitriptylinoxide, Amoxapine, Butriptyline, Clomipramine, Demexiptiline, Desipramine, Dibenzepin, Dimetracrine, Dothiepin, Doxepin, Fluacizine, Imipramine, Imipramine N-Oxide, Iprindole, Lofepramine, Melitracen, Metapramine, Nortriptyline, Noxiptilin, Opipramol, Pizotyline, Propizepine, Protriptyline, Quinupramine, Tianeptine, Trimipramine

Others such as Adrafinil, Benactyzine, Bupropion, Butacetin, Dioxadrol, Duloxetine, Etoperidone, Febarbamate, Femoxetine, Fenpentadiol, Fluoxetine, Fluvoxamine, Hematoporphyrin, Hypercinin, Levophacetoperane, Medifoxamine, Milnacipran, Minaprine, Moclobemide, Nefazodone, Oxaflozane, Piberaline, Prolintane, Pyrisuccideanol, Ritaserin, Ropxindole, Rubidium, Sulpiride, Tandospirone, Thozalinone, Tofenacin, Toloxatone, Tranylcypromine, L-Tryptophan, Venlafaxine, Viloxazine, Zimeldine

Antidiabetic

Biguanides such as Buformin, Metformin, Phenformin

Sulfonylurea Derivatives such as Acetohexamide, 1-Butyl-3-metanilylurea, Carbutamide, Chlorpropamide, Glibornuride, Gliclazide, Glimepiride, Glipizide, Gliquidone, Glisoxepid, Glyburide, Glybuthiazol(e), Glybuzole, Glyhexamide, Glymidine, Glypinamide, Phenbutamide, Tolazamide, Tolbutamide, Tolcyclamide

Others such as Acarbose, Calcium Mesoxalate, Miglitol, Repaglinide

ANTIDIARRHEAL such as Acetorphan, Acetyltannic Acid, Alkofanone, Aluminum Salicylates, Catechin, Difenoxin, Diphenoxylate, Lidamidine, Loperamide, Mebiquine, Trillium, Uzarin, Zaldaride

ANTIDIURETIC such as Desmopressin, Felypressin, Lypressin, Ornipressin, Oxycinchophen, Terlipressin, Vasopressin

ANTIESTROGEN such as Centchroman, Delmadinone Acetate, Tamoxifen, Toremifene

Antifungal (Antibiotics)

Polyenes such as Amphotericin-B, Candicidin, Dermostatin, Filipin, Fungichromin, Hachimycin, Hamycin, Lucensomycin, Mepartricin, Natamycin, Nystatin, Pecilocin, Perimycin

Others such as Azaserine, Griseofulvin, Oligomycins, Neomycin Undecylenate, Pyrrolnitrin, Siccanin, Tubercidin, Viridin

Antifungal (Synthetic)

Allylamines such as Butenafine, Naftifine

Imidazoles such as Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole Nitrate, Sertaconazole, Sulconazole, Tioconazole

Triazoles such as Fluconazole, Itraconazole, Saperconazole, Terconazole

Others such as Acrisorcin, Amorolfine, Biphenamine, Bromosalicylchloranilide, Buclosamide, Calcium Propionate, Chlophenesin, Ciclopirox, Cloxyquin, Coparaffinate, Diamthazole, Dihydrochloride, Exalamide, Flucytosine, Halethazole, Hexetidine, Loflucarban, Nifuratel, Potassium Iodide, Propionates, Propionic Acid, Pyrithione, Salicylanilide, Sulbentine, Tenonitrozole, Triacetin, Ujothion, Undecylenic Acid

ANTIGLAUCOMA such as Acetazolamide, Befunolol, Betaxolol, Brimonidine, Bupranolol, Carteolol, Dapiprazoke, Dichlorphenamide, Dipivefrin, Dorzolamide, Epinephrine, Latanoprost, Levobunolol, Methazolamide, Metrpranolol, Pilocarpine, Pindolol, Timolol, Unoprostone

ANTIGONADOTROPIN such as Danazol, Gestrinone, Paroxypropione

ANTIGOUT such as Allopurinol, Carpofen, Colchicine, Probenecid, Sulfinpyrazone

Antihistaminic

Alkylamine Derivatives such as Acrivastine, Bamipine, Brompheniramine, Chlorpheniramine, Dimethindene, Metron S, Pheniramine, Pyrrobutamine, Thenaldine, Tolpropamine, Triprolidine

Aminoalkyl Ethers such as Bietanautine, Bromodiphenhydramine, Carbinoxamine, Clemastine, Diphenylhydramine, Diphenlypyraline, Doxylamine, Embramine, Medrylamine, Moxastine p-Methyldiphenhydramine, Orphenadrine, Phenyltoloxamine, Setasine

Ethylenediamine Derivatives such as Alloclamide, Chloropyramine, Chlorothen, Histapyrrodine, Methafurylene, Methaphenilene, Methapyrilene, Pyrilamine, Talastine, Thenyldiamine, Thonzylamine, Tripelennamine, Zolamine

Piperazines such as Cetirizine, Chlorcyclizine, Cinnarizine, Clocinizine, Hydroxyzine

Tricyclics

Phenothiazines such as Ahistan, Etymemazine, Fenethazine, N-Hydroxyethylpromethazine, Isopromethazine, Mequitazine, Promethazine, Thiazinamium Methyl Sulfate

Other tricyclics such as Azatadine, Clobenzepam, Cyproheptadine, Deptropine, Isothipendyl, Loratadine

Others such as Antazoline, Astemizole, Azelastine, Cetoxime, Clemizole, Clobenztropine, Ebastine, Emedastine, Epinastine, Fexofenadine, Levocabastine, Mebhydroline, Phenindamine, Terfenadine, Tritoqualine

Antihyperlipoproteinemic

Aryloxyalkanoic Acid Derivatives such as Beclorbrate, Bazafibrate, Binifibrate, Ciprofibrate, Clinofibrate, Clofibrate, Clofibric Acid, Etonfibrate, Fenofibrate, Gemfibrozil, Nicofibrate, Pirifibrate, Ronifibrate, Simfibrate, Theofibrate

Bile Acid Sequesterants such as Cholestyramine Resin, Colestipol, Polidexide

HMG CoA Reductase Inhibitors such as Atorvastatin, Fluvastatin, Lovastatin, Pravastatin, Simvastatin

Nicotinic Acid Derivatives Acipimox, Aluminum Nicotinate, Niceritrol, Nicoclonate, Nicomol, Oxiniacic Acid

Thyroid Hormones/Analogs such as Etiroxate, Thyropropic Acid, Thyroxine

Others such as Acifran, Azacosterol, Benfluorex, beta-Benzalbutyramide, Carnitine, Chondroitin Sulfate, Clomestone, Detaxtran, Dextran Sulfate Sodium, 5,8,11,14,17-Eicosapentaenoic Acid, Eritadenine, Furazbol, Meglutol, Melinamide, Mytatrienediol, Ornithine, gamma-Oryzanol, Pantethine, Penataerythritol Tetraacetate, alpha-Phenylbutyramide, Phylate Acids and Salts, Pirozadil, Probucol, beta-Sitosterol, Sultosilic Acid, Tiadenol, Triparanol, Xenbucin

Antihypertensive

Benzothiadiazine Derivatives such as Althiazide, Bendroflumethiazide, Benzthiazide, Benzylhydrochlorothiazide, Buthiazide, Chlorothiazide, Chlorthalidone, Cyclopenthiazide, Cyclothiazide, Diazoxide, Epithiazide, Ethiazide, Fenquizone, Hydrochlorothiazide, Hydroflumethiazide, Indapamide, Methyclothiazide, Meticrane, Metolazone, Paraflutizide, Polythiazide, Quinethazone, Teclothiazide, Trichlormethiazide

N-Carboxyalkyl (peptide/lactam) Derivatives such as Alacepril, Benazepril, Captopril, Ceronapril, Cilazapril, Delapril, Enalapril, Enalaprilat, Fosinopril, Imidapril, Lisinopril, Moveltipril, Perindopril, Quinapril, Ramipril Spirapril, Temocarpril, Trandolapril

Guanidine Derivatives Bethanidine, Debrisoquin, Guanabenz, Guanacline, Guanadrel, Guanazodine, Guanethidine, Guanfacine, Guanochlor, Guanoxabenz, Guanoxan

Hydrazines/Phthalazines such as Budralazine, Cadralazine, Dihydralazine, Endralazine, Hydracarbazine, Hydralazine, Pheniprazine, Pildralazine, Todralazine

Imidazole Derivatives such as Clonidine, Lofexidine, Monoxidine, Phentolamine, Tiamenidine, Tolonidine

Quaternary Ammonium Compounds Azamethonium, Chlorisondamine, Hexamethonium, Pentacynium Bis(methyl sulfate), Pentamethonium, Pentolinium Tartate, Phenactopinium, Trimethidiunum Methosulfate

Quinazoline Derivatives such as Alfuzosin, Bunazosin Doxazosin, Prazosin, Terazosin, Trimazosin

Reserpine Derivatives such as Bietaserpine, Deserpidine, Rescinnamine, Reserpine, Syrosingopine

Sulfonamide Derivatives such as Ambuside, Clopamide, Furosemide, Quinethazone, Tripamide, Xipamide

Others such as Aimaline, gamma-Aminobutyric Acid, Bufeniode, Carmoxirole, Chlorthalidone, Cicletaine, Ciclosidomine, Clentiazem, Cryptenamine Tannates, Fantofarone, Fenoldopam, Flosequinan, Indoramin, Ketanserin, Levcromakalim, Metbutamate, Mecamylamine, Methyldopa, Methyl 4-Pyridyl Ketone Thiosemicarbarzone, Metolazone, Miberfradil, Minoxidil, Muzolimine, Naftopidil, Pargyline, Pempidine, Pinacidil, Piperoxan, Protoveratrines, Raubasine, Rescimetol, Saralasin, Semotiadil, Sodium Nitroprusside, Ticrynafen, Trimethaphan Camsylate, Tyrosinase, Urapidil

ANTIHYPERTHYROID such as 2-Amino-4-methylthiazole, 2-Aminothiazole, Carbimazole, 3,5-Dibromo-L-tyrosine, 3,5-Diiodotyrosine, Iodine, Methimazole, Methylthiouracil, Propylthiouracil, Sodium Perchlorate, Thibenzazoline, Thiobarbital, 2-Thiouracil

ANTIHYPOTENSIVE such as Amezinium Methyl Sulfate, Angiotensin Amide, Dimetofrine, Dopamine, Etifelmin, Etilefrin, Gepefrine, Metaraminol, Methoxamine, Midodrine, Norepinephrine, Pholedrine, Synephrine

ANTIHYPOTHYROID such as Levothyroxine, Liothyronine, Thyroid, Thyroidin, Thyroxine, Tiratricol, TSH

Anti-inflammatory (Nonsteroidal)

Aminoarylcarboxylic Acid Derivatives such as Enfenamic Acid, Etofenamate, Flufenamic Acid, Isonixin, Meclofenamic Acid, Mefanamic Acid, Niflumic Acid, Talniflumate, Terofenamate, Tolfenamic Acid

Arylacetic Acid Derivatives such as Aceclofenac, Acemetacin, Alclofenac, Amfenac, Amtolmetin Guacil, Bromfenac, Bufexamac, Cinmetacin, Clopirac, Diclofenac, Etodolac, Felbinac, Fenclozic Acid, Fentiazac, Glucametacin, Ibufenac, Indomethacin, Isofezolac, Isoxepac, Lonazolac, Metiazinic Acid, Mofezolac, Oxametacine, Pirazolac, Proglumetacin, Sulindac, Tiaramide, Tolmetin, Tropesin, Zomepirac

Arylbutyric Acid Derivatives such as Bumadizon, Butibufen, Fenbufen, Xenbucin

Arylcarboxylic Acids such as Clidanac, Ketorolac, Tinoridine

Arylpropionic Acid Derivatives such as Alminoprofen, Benoxaprofen, Bermoprofen, Bucloxic Acid, Carprofen, Fenoprofen, Flunoxaprofen, Flurbiprofen, Ibuprofen, Ibuproxam, Indoprofen, Ketoprofen, Loxoprofen, Naproxen, Oxaprozin, Piketoprofen, Pirprofen, Pranoprofen, Protizinic Acid, Suprofen, Tiaprofenic Acid, Ximoprofen, Zaltoprofen

Pyrazoles such as Difenamizole, Epirizole

Pyrazolones such as Apazone, Benzpiperylon, Feprazone, Mofebutazone, Morazone, Oxyphenbutazone, Phenylbutazone, Pipebuzone, Propyphenazone, Ramifenazone, Suxibuzone, Thiazolinobutazone

Salicylic Acid Derivatives such as Acetaminosalol, Aspirin, Benorylate, Bromosaligenin, Calcium Acetylsalicylate, Diflunisal, Etersalate, Fendosal, Gentisic Acid, Glycol Salicylate, Imidazole Salicylate, Lysine Acetylsalicylate, Mesalamine, Morpholine Salicylate, 1-Naphthyl Salicylate, Olsalazine, Parsalmide, Phenyl Acetylsalicylate, Phenyl Salicylate, Salacetamide, Salacetamide O-Acetic Acid, Salicylsulfuric Acid, Salsalate, Sulfasalazine

Thiazinecarboxamides such as Ampiroxicam, Droxicam, Isoxicam, Lornoxicam, Piroxicam, Tenoxicam

Others such as epsilon-Acetamidocaproic Acid, S-Adenosylmethionine, 3-Amino-4-hydroxybutyric Acid, Amixetrine, Bendazac, Benzydamine, alpha-Bisabolol, Bucolome, Difenpiramide, Ditazol, Emorfazone, Fepradinol, Guaiazulene, Nabumetone, Nimesulide, Oxaceprol, Paranyline, Perisoxal, Proquazone, Superoxide Dismutase, Tenidap, Zileuton

ANTIMALARIAL such as Acedapsone,, Amodiaquin, Arteether, Artemether, Artemisinin, Artesunate, Atovaquone, Bebeerine, Berberine, Chirata, Chlorguanide, Chloroquine, Chlorproguanil, Cinchona, Cinchonidine, Cinchonine, Cycloguanil, Gentiopicrin, Halofantrine, Hydroxychloroquine, Mefloquine Hydrochloride, 3-Methylarsacetin, Pamaquine, Plasmocid, Primaquine, Pyrimethamine, Quinacrine, Quinidine, Quinine, Quinocide, Quinoline, Sodium Arsenate, Diabasic

ANTIMIGRAINE such as Alpiropride, Dihydroergotamine, Dolasetron, Ergocornine, Ergocorninine, Ergocryptine, Ergot, Ergotamine, Flumedroxone Acetate, Fonazine, Lisuride, Methysergid(e), Oxetorone, Pizotyline, Sumatriptan

ANTINAUSEANT such as Acetylleucine Monoethanolamine, Alizapride, Azasetron, Benzquinamide, Bietanautine, Bromopride, Buclizine, Chlorpromazine, Clebopride, Cyclizine, Dimenhydrinate, Dipheniodol, Dolasetron, Domperidone, Granisetron, Meclizine, Methalltal, Metoclopramide, Metopimazine, Nabilone, Ondansteron, Oxypendyl, Pipamazine, Prochlorperazine, Scopolamine, Sulpiride, Tetrahydrocannabinols, Thiethylperazine, Thioproperazine, Trimethobenzamide, Tropisetron

Antineoplastic

Alkylating agents

Alkyl Sulfonates such as Busulfan, Improsulfan, Piposulfan

Aziridines such as Benzodepa, Carboquone, Meturedepa, Uredepa

Ethylenimines and Methylmelamines such as Altretamine, Triethylenemelamine, Triethylenephosphoramide, Triethylenethiophosphoramide

Nitrogen Mustards such as Chlorambucil, Chlornaphazine, Cyclophosphamide, Estramustine, Ifosfamide, Mechlorethamine, Mechlorethamine Oxide Hydrochloride, Melphalan, Novembichin, Perfosfamide, Phenesterine, Prednimustine, Trofosfamide, Uracil Mustard

Nitrosoureas Carmustine, Chlorozotocin, Fotemustine, Lomustine, Nimustine, Ranimustine

Others such as Dacarbazine, Mannomustine, Mitobronitol, Mitolactol, Pipobroman, Temozolomide

Antibiotics such as Aclacinomycins, Actinomycin F₁, Anthramycin, Azaserine, Bleomycins, Cactinomycin, Carubicin, Carzinophilin, Chromomycins, Dactinomycin, Daunorubicin, 6-Diazo-5-oxo-L-norleucine, Doxorubicin, Epirubicin, Idarubicin, Menogaril, Mitomycins, Mycophenolic Acid, Nogalamycin, Olivomycins, Peplomycin, Pirarubicin, Plicamycin, Porfiromycin, Puromycin, Streptonigrin, Streptozocin, Tubercidin, Zinostatin, Zorubicin

Antimetabolites

Folic Acid Analogs such as Denopterin, Edatrexate, Methotrexate, Piritrexim, Pteropterin, Tomudex®, Trimetrexate

Purine Analogs such as Cladribine, Fludarabine, 6-Mercaptopurine, Thiamiprine, Thioguanaine

Pyrimidine Analogs such as Ancitabine, Azacitidine, 6-Azauridine, Carmofur, Cytarabine, Doxifluridine, Emitefur, Enocitabine, Floxuridine, Fluororacil, Gemcitabine, Tegafur

Enzymes such as L-Asparaginase

Others such as Aceglatone, Amsacrine, Bisantrene, Defofamide, Demecolcine, Diaziquone, Elfornithine, Elliptinium Acetate, Etoglucid, Fenretinide, Gallium Nitrate, Hydroxyurea, Lonidamine, Miltefosine, Mitoguazone, Mitoxantrone, Mopidamol, Nitracrine, Pentostatin, Phenamet, Podophyllinicc Acid, 2-Ethythydrazide, Procarbazine, Razoxane, Sobuzoxane, Spirogermanium, Tenuazonic Acid, Triaziquone, 2,2′,2″-Trichlorotriethylamine, Urethan

Antineoplastic (Hormonal)

Androgens such as Calusterone, Dromostanolone, Epitiostanol, Mepitiostane, Testolactone

Antiadrenals such as Aminoglutethimide, Mitotane, Trilostane

Antiandrogens such as Bicalutamide, Flutamide, Nilutamide

Antiestrogens such as Droloxifene, Tamoxifen, Toremifene

Antineoplastic Adjunct

Folic Acid Replenisher such as Folinic Acid

ANTIPARKINSONIAN such as Amantadine, Benserazide, Bietanautine, Biperiden, Bromocriptine, Budipine, Carbidopa, Dexetimide, Diethazine, Droxidopa, Ethopropazine, Ethylbenzhydramine, Lazabemide, Levodopa, Mofegiline, Pergolide, Piroheptine, Pramipexole, Pridinol, Prodipine, Ropinirole, Selegiline, Talipexole, Terguride, Trihexyphenidyl Hydrochloride

ANTIPHEOCHROMOCYTOMA such as Metyrosine, Phenoxybenzamine, Phentolamine

ANTIPNEUMOCYSTIS such as Atovaquone, Effornithine, Pentamidine, Sulfamethoxazole

ANTIPROSTATIC HYPERTROPHY such as Epristeride, Finasteride, Gestonorone Caproate, Mepartricin, Osaterone, Oxendolone, Tamsulosin, Terazosin

ANTIPROTOZOAL (LEISHMANIA) such as Ethylstibamine, Hydroxystilbamidine, N-Methylglucamine, Pentamidine, Stilbamidine, Sodium Stibogluconate, Urea Stibamine

ANTIPROTOZOAL (TRICHOMONAS) such as Acetarsone, Aminitrozole, Anisomycin, Azanidazole, Furazolidone, Hachimycin, Lauroguadine, Mepartricin, Metronidazole, Nifuratel, Nifuroxime, Nimorazole, Secnidazole, Silver Picrate, Tenonitrozole, Tinidazole

ANTIPROTOZOAL (TRYPANOSOMA) such as Benznidazole, Eflornithine, Melarsoprol, Nifurtimox, Oxophenarsine, Pentamidine, Propamidine, Puromycin, Quinapyramine, Stilbamidine, Suramin Sodium, Trypan Red, Tryparasmide

ANTIPRURITIC such as Camphor, Cyproheptadine, Dichlorisone, Glycine, Halometasone, 3-Hydroxycamphor, Menthol, Mesulphen, Methdilazine, Phenol, Polidocanol, Spirit of Camphor, Thenaldine, Tolpropamine, Trimeprazine

ANTIPSORIATIC such as Acitretin, Ammonium Salicylate, Anthralin, 6-Azauridine, Bergapten(e), Calcipotriene, Chrysarobin, Etretinate, Lonapalene, Pyrogallol, Tacalcitol, Tazarotene

Antipsychotic

Butyrophenones such as Benperidol, Bromperidol, Droperidol, Fluanisone, Haloperidol, Melperone, Moperone, Pipamperone, Sniperone, Timiperone, Trifluperidol

Phenothiazines such as Acetophenazine, Butaperazine, Carphenazine, Chlorproethazine, Chlorpromazine, Clospirazine, Cyamemazine, Dixyrazine, Fluphenazine, Imiclopazine, Mepazine, Mesoridazine, Methoxypromazine, Metofenazate, Oxaflumazine, Perazine, Pericyazine, Perimethazine, Perphenazine, Piperacetazine, Pipotiazine, Prochlorperazine, Promazine, Sulforidazine, Thiopropazate, Thioridazine, Trifluoperazine, Triflupromazine

Thioxanthenes such as Chlorprothixene, Clopenthixol, Flupentixol, Thiothixene

Other Tricyclics such as Benzquinamide, Carpipramine, Clocapramine, Clomacran, Clothiapine, Clozapine, Mosapramine, Olanzapine, Opipramol, Prothipendyl, Seroquel®, Tetrabenazine, Zotepine

Others such as Buramate, Fluspirilene, Molindone, Penfluridol, Pimozide, Ziprasidone

ANTIPYRETIC such as Acetominophen, Acetaminosalol, Acetanilide, Alclofenac, Aluminum Bis(ascetylsalicylate), Aminochlorhenoxazin, Aminopyrine, Aspirin, Benorylate, Benzydamine, Berberine, Bermoprofen, para-Bromoacetanilide, Bufexamac, Bumadizon, Calcium Acetylsalicylate, Chlorthenoxazin(e), Choline Salicylate, Clidanac, Dihydroxyaluminum Acetylsalicylate, Dipyrocetyl, Dipyrone, Epirizole, Etersalate, Imidazole Salicylate, Indomethacin, Isofezolac, para-Lactophenetide, Lysine Acetylsalicylate, Magnesium Acetylsalicylate, Meclofenamic Acid, Morazone, Morpholine Salicylate, Naproxen, Mifenazone, 5′-Nitro-2′-propoxyacetanilide, Phenacetin, Phenicarbazide, Phenocoll, Phenopyrazone, Phenyl Acetylsalicyliate, Phenyl Salicylate, Pipebuzone, Propacetamol, Propyphenazone, Ramifenazone, Salacetamide, Ssalicylamide-O-Acetic Acid, Sodium Salicylate, Tetrandrine, Tinoridine

ANTIRICKETTSIAL such as p-Aminobenzoic Acid, Chloramphenicol, Tetracycline

ANTISEBORRHEIC such as Chloroxine, 3-O-Lauroylpyridoxol Diacetate, Piroctone, Pyrithione, Resorcinol, Selenium Sulfides, Tioxolone

Antiseptic

Guanidines such as Alexidine, Ambazone, Chlorhexidine, Picloxydine

Halogens/Halogen Compounds such as Bismuth Iodide Oxide, Bismuth Iodosubgallate, Bismuth Tribromophenate, Bornyl Chloride, Calcium Iodate, Chlorinated Lime, Cloflucarban, Iodic Acid, Iodine, Iodine Monochloride, Iodine Trichloride, Iodoform, Methenamine Tetraiodine, Oxychlorosene, Povidone-Iodine, Sodium Hypochlorite, Sodium Iodate, Symclosene, Triclocarban, Triclosan, Troclosene Potassium

Nitrofurans such as Furazolidone, 2-(Methoxymethyl)-5-Nitrofuran, Nidroxyzone, Nifuroxime, Nifurzide, Nitrofurazone

Phenols such as Acetomeroctol, Bithionol, Cadmium Salicylate, Carvacrol, Chloroxylenol, Clorophene, Creosote, Cresol, Fenticlor, Hexachlorophene, 1-Napthyl Salicylate, 2-Napthyl Salicylate, 2,4,6-Tribromo-m-cresol, 3′,4′,5-Trichlorosalicylanilide

Quinolines such as Aminoquinuride, Benzoxiquine, Broxyquinoline, Chloroxine, Chlorquinaldol, Cloxyquin, Ethylhydrocupreine, Euprocin, Halquinol, Hydrastine, 8-Hydroxquinoline Sulfate, Iodochlorhydroxyquin

Others such as Aluminum Acetate Solution, Aluminum Subacetate Solution, Aluminum Sulfate, 3-Amino-4-hydroxybutyric Acid, Boric Acid, Chlorhexidine, Chloroazodin, m-Cresyl Acetate, Cupric Sulfate, Dibromopropamidine, Ichthammol, Negatol, Noxythiolin, Octenidine, Ornidazole, beta-Propriolactone, alpha-Terpineol

ANTISPASMODIC such as Alibendol, Ambucetamide, Aminopromazine, Apoatropine, Bevonium Methyl Sulfate, Bietamiverine, Butaverine, Butropium, N-Butylscopolammonium

Bromide, Caroverine, Cimetropium, Cinnamedrine, Clebopride, Cyclonium Iodide, Difemerine, Diisopromine, Dioxaphetyl Butyrate, Diponium Bromide, Drofenine, Emepronium Bromide, Ethaverine, Etomidoline, Feclemine, Fenalamide, Fenoverine, Fenpiprane, Fenpiverinium Bromide, Fentonium Bromide, Flavoxate, Flopropione, Gluconic Acid, Hydramitrazine, Hymecromone, Leiopyrrole, Mebeverine, Moxaverine, Nafiverine, Octamylamine, Octaverine, Pentapiperide, Phenamacide, Phloroglucinol, Pinaverium, Piperilate, Pipoxolan Hydrochloride, Pramiverin, Prifinium Bromide, Propyromazine, Prozapine, Racefemine, Rociverine, Sintropium Bromide, Spasmolytol, Sultroponium, Tiemonium Iodide, Tigloidine, Tiquizium Bromide, Tiropramide, Trepibutone, Tricromyl, Trifolium, Trimebutine, N,N-1Trimethyl-3,3-diphenylpropylamine, Tropenzile, Trospium Chloride, Xenotropium Bromide

ANTITHROMBOTIC such as Argatroban, Cilostazol, Clopidrgrel, Cloricromen, Dalteparin, Daltroban, Defibrotide, Enoxaparin, Indobufen, Iloprost, Integrelin, Isbogrel, Lamifiban, Lamoparan, Nadroparin, Ozagrel, Picotamide, Plafibride, Reviparin Sodium, Ridogrel, Sulfinpyrazone, Taprostene, Ticlopidine, Tinzaparin, Tirofiban, Triflusal

ANTITUSSIVE such as Allocamide, Amicibone, Benproperine, Benzonatate, Bibenzonium, Bromoform, Butamirate, Butethamate, Caramiphen Ethanedisulfonate, Carbetapentane, Chlophedianol, Clobutinol, Cloperastine, Codeine, Codeine Methyl Bromide, Codeine N-Oxide, Codeine Phosphate, Codeine Sulfate, Cyclexanone, Dextromethorphan, Dihydrocodeine, Dihydrocodeinone Enol Acetate, Dimemorfan, Dimethoxanate, Dropropizine, Drotebanol, Eprazinone, Ethyl Dibunate, Ethylmorphine, Fominoben, Guiaiapate, Hydrocodone, Isoaminile, Levopropoxyphene, Morclofone, Narceine, Normethadone, Noscapine, Oxeladin, Oxolamine, Pholcodine, Picoperine, Pipazethate, Piperidione, Prenoxdiazine, Racemethorphan, Sodium Dibunate, Tipepidine, Zipeprol

ANTIULCERATIVE such as Aceglutamide Aluminum Complex, epsilon-Acetamidocaproic Acid Zinc Salt, Acetoxolone, Aldioxa, Arbaprostil, Benexate Hydrochloride, Carbenoxolone, Cetraxate, Cimetidine, Colloidal Bismuth Subcitrate, Ebrotidine, Ecabet, Enprostil, Esaprazole, Famotidine, Gefarnate, Guaiazulene, Irsogladine, Lansoprazole, Misoprostol, Nizatidine, Omeprazole, Ornoprostil, gamma-Oryzanol, Pantoprazole, Pifarnine, Pirenzepine, Plaunotol, Polaprezinc, Rabeprazole, Ranitidine, Rebamipide, Rioprostil, Rosaprostol, Rotraxate, Roxatidine Acetate, Sofaicone, Spizofurone, Sucralfate, Telenzepine, Teprenone, Trimoprostil, Thrithiozine, Troxipide, Zolimidine

ANTIUROLITHIC such as Acetohydroxamic Acid, Allopurinol, Potassium Citrate, Succinimide

ANTIVENIN such as Lyovac Antivenin

Antiviral

Purines/Pyrimidinones such as Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidrarbine, Zalcitabine, Zidovudiine

Others such as Acemannan Acetylleucine Monoethanolamine, Amantadine, Amidinomycin, Delaviridine, Foscarnet Sodium, Indinavir, Interferon (alpha, beta, gamma), Kethoxal, Lysozyme, Methisazone, Moroxydine, Nevirapine, Podophyllotoxin, Ribavirin, Rimantadine, Ritonavir, Saquinavir, Stallimycin, Statolon, Tromantadine, Xenazoic Acid

Anxiolytic

Arylpiperazines such as Buspirone, Enciprazine, Flesinoxan, Ipsapirone, Lesopitron, Tandospirone

Benzodiazepine Derivatives Alprazolam, Bromazepam, Camazepam, Chlordiazepoxide, Clobazam, Clorazepate, Chotiazepam, Cloxazolam, Diazepam, Ethyl Loflazepate, Etizolam, Fluidazepam, Flutazolam, Flutoprazepam, Halazepam, Ketazolam, Lorazepam, Loxapine, Medazepam, Metaclazepam, Mexazolam, Nordazepam, Oxazepam, Oxazolam, Pinazepam, Prazepam, Tofisopam

Carbamates such as Cyclarbamate, Emylcamate, Hydroxyphenamate, Meprobamate, Phenprobamate, Tybamate

Others Abecarnil, Alpidem, Benzoctamine, Captodiamine, Chlormezanone, Etifoxine, Fluoresone, Glutamic Acid, Hydroxyzine, Mecloralurea, Mephenoxalone, Oxanamide, Pazinaclone, Suriclone

BENZODIAZEPINE ANTAGONIST such as Flumazenil

Bronchodilator

Ephedrine Derivatives such as Albuterol, Bambuterol, Bitolterol, Carbuterol, Clenbuterol, Clorprenaline, Dioxethedrine, Ephedrine, Epinephrine, Eprozinol, Etafedrine, Ethylnorepinephrine, Fenoterol, Formoterol, Hexoprenaline, Isoetharine, Isoproterenol, Mabuterol, Metaproterenol, N-Methylephedrine, Pirbuterol, Procaterol, Protokylol, Reproterol, Rimiterol, Salmeterol, Soterenol, Terbutaline, Tulobuterol

Quaternary Ammonium Compounds such as Bevonium Methyl Sulfate, Flutropium Bromide, Ipratropium Bromide, Oxitropium Bromide, Tiotropium Bromide

Xanthine Derivatives such as Acefylline, Acefylline Piperazine, Ambuphylline, Aminophylline, Bamifylline, Choline Theophyllinate, Doxofylline, Dyphylline, Etamiphyllin, Etofylline, Guaithylline, Proxyphylline, Theobromine, 1-Theobromineacetic Acid, Theophylline

Others such as Fenspiride, Medibazine, Methoxyphenanime, Tretoquinol

Calcium Channel Blocker

Arylalkylamines such as Bepridil, Clentiazen, Diltiazem, Fendiline, Gallopamil, Mibefradil, Prenylamine, Semotiadil, Terodiline, Verapamil

Dihydropyridine Derivatives such as Amlodipine, Aranidipine, Barnidipine, Benidipine, Cilnidipine, Efonidipine, Elgodipine, Felodipine, Isradipine, Lacidipine, Lercanidipine, Manidipine, Nicardipine, Nifedipine, Nilvadipine, Nimodipine, Nisoldipine, Nitrendipine

Piperazine Derivatives such as Cinnarizine, Flunarizine, Lidoflazine, Lomerizine

Others such as Bencyclane, Etafenone, Fantofarone, Perhexiline

CALCIUM REGULATOR such as Calcifediol, Calcitonin, Calcitriol, Dihydrotachysterol, Elcatonin, Ipriflavone, Parathyroid Hormone, Teriparatide Acetate

CARDIOTONIC such as Acetfylline, Acetyldigititoxins, 2-Amino-4-picoline, Amrinone, Benfurodil Hemisuccinate, Buclasdesine, Camphotamide, Convallatoxin, Cymarin, Denopamine, Deslanoside, Digitalin, Digitalis, Digitoxin, Digoxin, Dobutamine, Docarpamine, Dopamine, Dopexamine, Enoximone, Erythrophleine, Fenalcomine, Gitalin, Gitoxin, Glycocyamine, Heptaminol, Hydrastinine, Ibopamine, Lanotodises, Loprinone, Milrinone, Neriifolin, Oleandrin, Ouabain, Oxyfedrine, Pimobendan, Prenalterol, Proscillaridin, Resibufogenin, Scillaren, Scillarenin, Strophanthin, Sulmazole, Theobromine, Vesnarinone, Xamoterol

CHELATING AGENT such as Deferoxazmine, Ditiocarb Sodium, Edetate Calcium Disodium, Edetate Disodium, Edeate Sodium, Edetate Trisodium, Penicillamine, Pentetate Calcium Trisodium, Pentectic Acid, Succimer, Trientine

Cholecystokinin Antagonist (CCK Antagonist)

CHOLELITHOLYTIC AGENT such as Chenodiol, Methyl tert-Butyl Ether, Monooctanoin, Ursodiol

CHOLERETIC such as Alibendol, Anethole Trithion, Azintamide, Cholic Acid, Cicrotoic Acid, Clanobutin, Cyclobutyrol, Cyclovalone, Cynarin(e), Dehydrocholic Acid, Deoxycholic Acid, Dimecrotic Acid, alpha-Ethylbenzyl Alcohol, Exiproben, Febuprol, Fencibutirol, Fenipentol, Florantyrone, Hymecromone, Menbutone, 3-(o-Methoxyphenyl)-2-phenylacrylic Acid, Metochalcone, Moquizone, Osalmid, Ox Bile Extract, 4,4′-Oxydi-2-butanol, Piprozolin, 4-Salicyloylmorpholine, Sincalide, Taurocholic Acid, Tocamphyl, Trepibutone, Vanitiolide

CHOLINERGIC such as Aceclidine, Acetylcholine, Acetylcholide, Aclatonium Napadisilate, Benzpyrinium Bromide, Bethanechol, Carbachol, Carpronium, Demecarium, Dexpanthenol, Diisopropyl Paraoxon, Echothiophate, Edrophomium, Eptastigmine, Eseridine, Furtrethonium, Isoflurophate, Methacholine Chloride, Muscarine, Neostigmine, Oxapropanium, Physostigmine, Pyridostigmine, Xanomeline

CHOLINESTERASE INHIBITOR such as Ambenonium, Distigmine, Eptastigmine, Galanthamine

CHOLINESTERASE REACTIVATOR such as Asoxime, Obidoximine, Pralidoxime

CNS STIMULANT/AGENT such as Amineptine, Amphetimine, Amphetaminil, Bemegride, Benzphetamine, Brucine, Caffeine, Chlorphentermine, Clortermine, Coca, Deanol, Demanyl Phosphate, Dexoxadrol, Dextroamphetamine Sulfate, Diethylpropion, N-Ethylamphetamine, Ethamivan, Etifelmin, Etryptamine, Fencamfamine, Fenethylline, Fenozolone, Flurothyl, Hexacyclonate Sodium, Homocamfin, Mazindol, Mefexamide, Methamphetamine, Methylphenidate, Modafinil, Nikethamide, Pemoline, Pentylenetetrazole, Phenidimetrazine, Phenmetrazine, Phentermine, Picrotoxin, Pipradrol, Prolintane, Pyrovalerone, Tetrahydrobenzothienopyridines

DECONGESTANT such as Amidephrine, Cafaminol, Cyclopentamine, Ephedrine, Epinephrine, Fenoxazoline, Indanazoline, Metizoline, Naphazoline, Nordefrin, Octodrine, Oxymetazoline, Phenylephrine, Phenylpropanolamine, Phenylpropylmethylamine, Propyhexedrine, Pseudoephredrine, Tetrahydrolzoline, Tramazoline, Tuaminoheptane, Tymazoline, Xylometazoline

DENTAL CARRIES PROPHYLACTIC such as Sodium Fluoride

DEPIGMENTOR such as Hydroquinine, Hydroquinone, Monobenzone

Diuretic

Organomercurials such as Chlormerodrin, Meralluride, Mercamphamide, Mercaptomerin Sodium, Mercumallylic Acid, Mercumatilin Sodium, Mercurous Chloride, Mersalyl

Purines such as Acefylline, 7-Morpholinomethyltheophylline, Pamabrom, Protheobromine, Theobromine

Steroids such as Canrenone, Oleandrin, Spironolactone

Sulfonamide Derivatives such as Acetazolmide, Ambuside, Azosemide, Bumetanide, Butazolamide, Chloraminophenamide, Clofenamide, Clopamide, Clorexolene, Disulfamide, Ethoxzolamide, Furosemide, Mefruside, Methazolamide, Piretanide, Torasemide Tripamide, Xipamide

Uracils such as Aminometradine, Amisometradine

Others such as Amanozine, Amiloride, Arbutin, Chlorazanil, Ethacrynic Acid, Etozolin, Hydracarbazine, Isosorbide, Mannitol, Metochalcone, Muzolimine, Perhexiline, Triamterene, Urea

DOPAMINE RECEPTOR AGONIST such as Bromocriptine, Cabergoline, Carmoxirole, Dopexamine, Fenoldopam, Ibopamine, Lisuride, Pergolide, Pramipexole, Quinagolide, Ropinirole, Roxindole, Talipexole

ECTOPARASITICIDE such as Amitraz, Benzyl Benzoate, Carbaryl, Crotamiton, DDT, Dixanthogen, Lime Sulfurated Solution, Lindane, Malathion, Mercuric Oleate, Mesulfen, Sulfiram, Sulphur (Pharmaceutical)

Enzyme

Digestive such as Amylase, Lipase, Pancrelipase, Pepsin, Rennin

Penicillin Inactivating such as Penicillinase

Proteolytic such as Collagenase, Chymopapain, Chymotrypsins, Papain, Trypsin

ENZYME INDUCER (HEPATIC) such as Flumecinol

Estrogen

Nonsteroidal such as Benzestrol, Broparoestrol, Chlorotrianisene, Dienestrol, Diethylstilbestrol, Dimestrol, Fosfestrol, Hexestrol, Methallenestril, Methestrol

Steroidal such as Colpormon, Conjugated Estrogenic Hormones, Equilenin, Equilin, Estradiol, Estriol, Estrone, Ethinyl Estradiol, Mestranol, Moxestrol, Mytatrienediol, Quinestradiol, Quinestrol

GASTRIC SECRETION INHIBITOR such as Enterogastrone, Octreotide, Telenzepine

GLUCOCORTICOID such as 21-Acetoxyprefnenolone, Alclometasone, Algestone, Amcinonide, Beclomethasone, Betamethasone, Budesonide, Chloroprednisone, Clobetasol, Clobetasone, Clocortolone, Cloprednol, Corticosterone, Cortisone, Cortivazol, Deflazacort, Desonide, Desoximetasone, Dexamethasone, Diflorasone, Diflucortolone, Difluprednate, Enoxolone, Fluazacort, Flucloronide, Flumethasone, Flunisolide, Fluocinolone Acetonide, Fluocinonide, Fluocortin Butyl, Fluocortolone, Fluorometholone, Fluperolone Acetate, Fluprednidene Acetate, Fluprednisolone, Flurandrenolide, Fluticasone Propionate, Formocortal, Halcinonide, Halobetasol Propionate, Halometasone, Halopredone Acetate, Hydrocortamate, Hydrocortisone, Loteprednol Etabonate, Mazipredone, Medrysone, Meprednisone, Methyolprednisolone, Mometasone Furcate, Paramethasone, Prednicarbate, Prednisolone, Prednisolone 25-Diethylaminoacetate, Prednisone Sodium Phosphate, Prednisone, Prednival, Prednylidene, Rimexolone, Tixocortol, Triamcinolone, Triamcinolone Acetonide, Triamcinolone Benetonide, Triamcinolone Hexacetonide

GONAD-STIMULATING PRINCIPLE such as Buserelin, Chorionic Gonadotropin, Clomiphene, Cyclofenil, Epimestrol, FSH, LH, LH-RH

GONADOTROPIC HORMONE such as LH, PMSG

GROWTH HORMONE INHIBITOR such as Octreotide, Somatostatin

GROWTH HORMONE RELEASING FACTOR such as Semorelin

GROWTH STIMULANT such as Somatotropin

HEMOLYTIC such as Phenylhydrazine

HEPARIN ANTAGONIST such as Hexadimethrine

HEPATOPROTECTANT such as S-Adenosulmethionine, Betaine, Catechin, Citolone, Malotilate, Methionine, Orazamide, Phosphorylcholine, Protoporphyrin IX, Silymarin-Group, Thiotic Acid, Timonacic, Tiopronin

IMMUNOMODULATOR such as Acemannan, Amiprilose, Bucillamine, Ditiocarb Sodium, Imiquimod, Inosine Pranobex, Interferon (alpha, beta, gamma), Lentinan, Levamisole, Macrophage Colony Stimulating Factor, Pidotimod, Platonin, Procodazole, Propagermanium, Romurtide, Thymomodulin, Thymopentin, Ubenimex

IMMUNOSUPPRESSANT such as Azathioprine, Brequinar, Cyclosporins, Gusperimus, 6-Mercaptopurine, Mizoribine, Rapamycin

ION EXCHANGE RESIN such as Carbacrylic Resins, Cholestyramine Resin, Colestipol, Polidexide, Resodec, Sodium Polystyrene Sulfonate

LACTATION STIMULATING HORMONE such as Prolactin

LH-RH AGONIST such as Buserelin, Deslorelin, Goserelin, Histrelin, Leuprolide, Nafarelin, Triptorelin

LIPOTROPIC such as N-Acetylmethionine, Choline Chloride, Choline Dehydrocholate, Choline Dihydrogen Citrate, Inositol, Lecithin, Methionine

LUPUS ERYTHEMATOSUS SUPPRESSANT such as Bismuth Sodium Triglycollamate, Bismuth Subsalicylate, Chloroquine, Hydroxychloroquine

MINERALOCORTICOID such as Aldosterone, Deoxycorticosterone, Fludrocortisone

MIOTIC such as Carbachol, Neostigmine, Physostigmine, Pilocarpine

MONOAMINE OXIDASE INHIBITOR such as Iproclozide, Iproniazid, Isocarboxazid, Lazabemide, Mefegiline, Meclobemide, Octamoxin, Pargyline, Phenelzine, Phenoxypropazine, Pivalylbenzhydrazine, Prodipine, Selegiline, Toloxatone, Tranylcypromine

MUCOLYTIC such as Acetylcysteine, Bromhexine, Carbocysteine, Domiodol, Erdosteine, Letosteine, Lysozyme, Mecysteine, Mesna, Sobrerol, Stepronin, Tiopronin, Tyloxapol

MUSCLE RELAXANT (SKELETAL) such as Afloqualone, Alcuronium, Atracurium Besylate, Baclofen, Benzoctamine, Benzoquinonium, C-Calebassine, Carisoprodol, Chlormezanone, Chlorphenesin Carbamate, Chlorphenesin, Chlorproethazine, Chlozoxazone, Curare, Cyclobamate, Cyclobenzaprine, Dantrolene, Decamethonium, Diazepam, Doxacurium Chloride, Eperisone, Fazadinium, Flumetramide, Gallamine Triethiodide, Hexacarbacholine, Hexafluorenium, Idrocilamide, Inaperisone, Lauexium Methyl Sulfate, Leptodactyline, Memantine, Mephenesin, Mephenoxalone, Metaxalone, Methocarbamol, Metocurine Iodide, Mivacurium Chloride, Nimetazepam, Orphenadrine, Pancuronium, Phenprobamate, Phenyramidol, Pipecurium, Promoxolane, Quinine, Rocuronium, Styramate, Succinylcholine, Suxethonium Bromide, Tetrazepam, Thiocolchicoside, Tizanidine, Tolperisone, Tubocurarine, Vecuronium, Zoxolamine

NARCOTIC ANTAGONIST such as Amiphenazole, Cyclazocine, Levallorphan, Nalmafene, Nalorphine, Naloxone, Naltrexone

NEUROPROTECTIVE such as Riluzole

NOOTROPIC such as Aceglutamide, Acetylcarnitine, Aniracetam, Besipridine, Bifemalane, Choline Alfoscerate, Exifone, Fipexide, Idebenone, Indeloxazune, Nebracetam, Nefiracetam, Nizofenone, Oxiracetam, Piracetam, Pramiracetam, Propentofylline, Pyritinol Sabeluzole, Tacrine, Velnacrine, Vinconate, Xanomeline

OPHTHALMIC AGENT such as 15-ketoprostaglandins

OVARIAN HORMONE such as Relaxin

OXYTOCIC such as Carboprost, Cargutocin, Deaminooxytocin, Ergonovine, Gemeprost, Methylergonovine, Oxytocin, Pituitary (Posterior), Prostaglandin E₂, Prostaglandin F₂ₐ, Sparteine

PEPSIN INIBITOR such as Sodium Amylosulfate

PERISTALTIC STIMULANT such as Cinitapride, Cisapride, Fedotozine, Loxiglumide

PROGESTOGEN such as Allylestrenol, Anagestone, Chlormadinone Acetate, Delmadinone Acetate, Demegestone, Desogestrel, Dimethisterone, Drospirenone, Dydrogesterone, Ethisterone, Ethynodiol, Flurogestone Acetate, Gestodene, Gestonorone Caproate, 17-Hydroxy-16-methylene-progesterone, 17 alpha-Hydroxyprogesterone, Lynestrenol, Medrogestone, Medroxyprogesterone, Megestrol Acetate, Melengestrol, Norethindrone, Norethynodrel, Norgesterone, Norgestimate, Norgestrel, Norgestrienone, Norvinisterone, Pentagestrone, Progesterone, Promegestone, Trengestone

PROLACTIN INHIBITOR such as Bromocriptine, Cabergoline, Lisuride, Metergoline, Quinagolide, Terguride

PROSTAGLANDIN/PROSTAGLANDIN ANALOG such as Arbaprostil, Bemeprost, Carboprost, Enprostil, Gemeprost, Latanoprost, Limaprost, Misoprostol, Ornoprostil, Prostacyclin, Prostaglandin E₁, Prostaglandin E₂, Prsotaglandin F₂ₐ, Rioprostil, Rosaprostol, Sulprostone, Trimoprostil, Unoprostone

PROTEASE INHIBITOR such as Aprotinin, Camostat, Gabexate, Nafamostat, Urinastatin

RESPIRATORY STIMULANT such as Almitrine, Bemegride, Cropropamide, Crotethamide, Dimefline, Dimorpholamine, Doxapram, Ethamivan, Forminoben, Lobeline, Mepixanox, Nikethamide, Picotoxin, Pimeclone, Pyridofylline, Sodium Succinate, Tacrine

SCLEROSING AGENT such as Ethanolamine, Ethylamine, 2-Hexyldecanoic Acid, Polidocanol, Sodium Ricinoleate, Sodium Tetradecyl Sulfate, Tribenoside

Sedative/hypnotic

Acyclic Ureides such as Acecarbromal, Apronalide, Bomisovalum, Capuride, Carbromal, Ectylurea

Alcohols such as Chlorhexadol, Ethchlorvynol, Meparfynol, 4-Methyl-5-thiazoleethanol, tert-Pentyl Alcohol, 2,2,2-Trichloroethanol

Amides such as Butoctamide, Diethylbromoacetamide, Isovaleryl Diethylamide, Niaprazine, Trimetozine, Zolpidem, Zopiclone

Barbituric Acid Derivatives such as Allobarbital, Amobarbital, Aprobarbital, Barbital, Brallabarbital, Butabarbital Sodium, Butalbital, Butallylonal, Butethal, Carbubarb, Cyclobarbital, Cyclopentobarbital, Enallylpropymal, 5-Furfuryl-5-isopropylbarbituric Acid, Heptabarbital, Hexethal Sodium, Hexobarbital, Mephobarbital, Methitural, Narcobarbital, Nealbarbital, Pentobarbital Sodium, Phenallymal, Phenobarbital, Phenylmethylbarbituric Acid, Propallylonal, Proxibarbal, Reposal, Secobarbital Sodium, Talbutal, Tetrabarbital, Vinbarbital Sodium, Vinylbital

Benzodiazepine Derivatives such as Brotizolam, Cinolazepam, Doxefazepam, Estazolam, Flunitrazepam, Flurazepam, Haloxazolam, Loprazolam, Lormetazepam, Nitrazepam, Quazepam, Temazepam, Triazolam

Bromides such as Ammonium Bromide, Calcium Bromide, Calcium Bromolactobionate, Lithium Bromide, Magnesium Bromide, Potassium Bromide, Sodium Bromide

Carbamates such as Carfimate, Ethinamate, Hexapropymate, Novonal, Tricholorourethan

Chloral Derivatives such as Carbocloral, Chloral Betaine, Chloral Formamide, Chloral Hydrate, Dichloralphenazone, Pentaerythritol Chloral, Triclofos

Piperidinediones such as Glutethimide, Methyprylon, Piperidione, Pyrithyldione, Thalidomide

Quinazolone Derivatives such as Etaqualone, Mecloqualone, Methaqualone

Others such as Acetal, Acetophenone, Aldol, Ammonium Valerate, Amphenidone, d-Bornyl alpha-Bromoisovalerate, d-Bornyl Isovalerate, Bromoform, Calcium 2-Ethylbutanoate, alpha-Chlorolose, Clomethiazole, Cypripedium, Doxylamine, Etodroxizine, Etomidate, Fenadiazole, Homofenazine, Hydrobromic Acid, Mecloxamine, Menthyl Valerate, Opium, Paraldehyde, Perlapine, Propiomazine, Rilmazafone, Sodium Oxybate, Sulfonethylmethane, Sulfonmethane

THROMBOLYTIC such as Anistreplase, Plasmin, Pro-Urokinase, Streptokinase, Tissue Plasminogen Activator, Urokinase

Thyrotropic Hormone such as TRH, TSH

URICOSURIC such as Benzbromarone, Ethebenecid, Orotic Acid, Oxycinchophen, Probenecid, Sulfinpyrazone, Zoxazolamine

VASODILATOR (CEREBRAL) such as Bencyclane, Cinnarizine, Citicoline, Cyclandelate, Ciclonicate, Diisopropylamine Dichloractetate, Eburnamonine, Fasudil, Fenoxedil, Flunarizine, Ibudilast, Ifenprodil, Lomerizine, Nafronyl, Nicametate, Nicergoline, Nimodipine, Papaverine, Pentifylline, Tinofedrine, Vincamine, Vinpocetine, Viquidil

VASODILATOR (CORONARY) such as Amotriphene, Bendazol, Benfurodil Hemisuccinate, Benziodcarone, Chloacizine, Chromonar, Clobenfurol, Clonitrate, Dilazep, Dipyridamole, Droprenilamine, Efloxate, Erythrityl Tetranitrate, Etafenone, Fendiline, Floredil, Ganglefene, Heart Muscle Extract, Hexestrol Bis(.beta.-diethylaminoethyl ether), Hexobendine, Itramin Tosylate, Khellin, Lidoflazine, Mannitol Hexanitrate, Medibazine, Nitroglycerin, Pentaerythritol Tetranitrate, Pentrinitrol, Perhexiline, Pimefylline, Prenylamine, Propatyl Nitrate, Pyridofylline, Trapidil, Tricromyl, Trimetazidine, Trolnitrate Phosphate, Visnadine

VASODILATOR (PERIPHERAL) such as Aluminum Nicotinate, Bamethan, Bencyclane, Betahistine, Bradykinin, Brovincamine, Bufeniode, Buflomedil, Butalamine, Cetiedil, Ciclonicate, Cinepazide, Cinnarizine, Cyclandelate, Diisopropylamine Dichloroacetate, Eledoisin, Fenoxedil, Hepronicate, Iloprost, Inositol Niacinate, Isoxsuprine, Kallidin, Kallikrein, Moxisylyte, Nafronyl, Nicergoline, Nicofuranose, Nicotinyl Alcohol, Nylidrin, Pentifylline, Pentoxifylline, Prostaglandin E₁, Piribedil, Suloctidil, Tolazoline, Xanthinal Niacinate

VASOPROTECTANT such as Benzarone, Bioflavonoids, Chromocarb, Clobeoside, Diosmin, Dobesilate Calcium, Escin, Folescutol, Leucocyanidin, Metescufylline, Quercetin, Rutin, Troxerutin

VITAMIN/VITAMIN SOURCE/EXTRACTS such as Vitamins A, B, C, D, E, and K and derivatives thereof

VULNERARY such as Acetylcysteine, Allantoin, Asiaticoside, Cadexomer Iodine, Chitin, Dextranomer, Oxaceprol, Tocoretinate

The above list of pharmaceutical agents is based upon the list provided in The Merk Index, 21th Edition, Merck & Co. Rahway, N.J. (1996). Moreover, the above drugs may be used either in the free form or, if capable of forming salts, in the form of a salt with a suitable acid or base; if the drug has a carboxyl group, its esters may also be employed.

The preferred embodiments described herein are illustrative only, and although the examples given include much specificity, they are intended as illustrative of only a few possible embodiments of the invention. Other embodiments and modifications will, no doubt, occur to those skilled in the art. The examples given should only be interpreted as illustrations of some of the preferred embodiments of the invention.

What is claimed is:

1. An active agent composition using liposome beads for topical application comprising:
a. preparing a liposomal suspension of multilamellar vesicles wherein the liposomal suspension of multilamellar vesicles encapsulate at least one active agent,
b. mixing the liposomal suspension of multilamellar vesicles into a bonding solution resulting in a liposomal first solution,
c. introducing an aliquot of the liposomal first solution into a second solution, the second solution containing at least one inorganic salt, wherein the aliquot of the liposomal first solution develops a hardened surface upon contact with the second solution to form a liposome bead, the hardened surface having a yield strength of 1 to 4 grams per cubic millimeter,
d. repeating step C to form a plurality of liposome beads,
e. aggregating the plurality of liposome beads, and
f. washing the plurality of liposome beads with a chemically inert solution.

2. The composition of claim 1, wherein the liposomal suspension of multilamellar vesicles is derived from a phospholipid.

3. The composition of claim 1, wherein the at least one active agent is from a class of compounds selected from the group consisting of antifungal drugs, anti-inflammatory drugs, anti-arthritic drugs, corticosteroids, vitamins, whitening agents, nitrous oxide, moisturizers, anabolic drugs, analgesic drugs, anesthetic drugs, anti-asthmatic drugs, antibacterial drugs, antihistaminic drugs, anti-neoplastic drugs, anti-parasitic drugs, vasodilator drugs, vasoconstrictor drugs, anti-tumor drugs, anti-viral drugs, anti-seborrheic drugs, anti-vertigo drugs, deliverance of toxins, deliverance of hormones, delivery of nicotine, compounds for anti-cold, compounds for release of heat, compounds for prevention of contact dermatitis, compounds for prevention of irritants, minerals, amino acids, lipids, herbs and metabolite supplements.

4. The composition of claim 1, wherein the at least one active agent is in an amount from about 0.01 to about 5 weight percent based on a total weight of the liposomal suspension of multilamellar vesicles.

5. The composition of claim 1, wherein the bonding solution is a physical bonding solution

6. The composition of claim 1, wherein the bonding solution is a physiochemical bonding solution.

7. The composition of claim 1, wherein the bonding solution is selected from the group consisting of aragrose, cellulose, sodium alginate, chitosans, and polymeric substances.

8. The composition of claim 1, wherein the step of introducing the aliquot of the liposomal first solution into the second solution is performed by regulating a flow of the liposomal first solution through an orifice.

9. The composition of claim 1, wherein the at least one inorganic salt is selected from the group consisting of calcium chloride, calcium sulfate, calcium carbonate, magnesium chloride, magnesium sulfate, barium chloride, barium sulfate and sodium hydroxide.

10. The composition of claim 1, wherein the at least one inorganic salt is in an amount from about 1 to 2 weight percent of the second solution.

11. The composition of claim 1, wherein the step of aggregating the plurality of liposome beads is performed by physical separation.

12. A liposome encapsulated composition for topical application comprising:
a therapeutically effective amount of at least one active agent encapsulated in a liposome suspension of multilamellar vesicles in an amount from about 0.01 to about 5 weight percent based on a total weight of the liposomal suspension of multilamellar vesicles in admixture with a bonding solution,
wherein the liposomal suspension of multilamellar vesicles in admixture with the bonding solution is encapsulated within a hardened bead shell.

13. The liposome encapsulated composition of claim 12, wherein the hardened bead shell is formed by introducing an aliquot of the liposomal suspension of multilamellar vesicles in admixture with the bonding solution into a second solution, the second solution containing at least one inorganic salt.

14. The liposome encapsulated composition of claim 13, wherein the at least one inorganic salt is selected from the group consisting of calcium chloride, calcium sulfate, calcium carbonate, magnesium chloride, magnesium sulfate, barium chloride, and barium sulfate.

15. The liposome encapsulated bead composition of claim 13, wherein the at least one inorganic salt is in an amount from about 1 to 2 weight percent of the second solution.

16. The liposome encapsulated composition of claim 12, wherein the hardened bead shell has a yield strength of 1 to 4 grams per cubic millimeter.

17. The liposome encapsulated composition of claim 12, wherein the at least one active agent is in solid form at ambient temperatures and pressures.

18. The liposome encapsulated composition of claim 12, wherein the at least one active agent is in liquid form at ambient temperatures and pressures.

19. The liposome encapsulated composition of claim 12, wherein the at least one active agent is from a class of compounds selected from the group consisting of antifungal drugs, anti-inflammatory drugs, anti-arthritic drugs, corticosteroids, vitamins, whitening agents, nitrous oxide, moisturizers, anabolic drugs, analgesic drugs, anesthetic drugs, anti-asthmatic drugs, antibacterial drugs, antihistaminic drugs, anti-neoplastic drugs, antiparasitic drugs, vasodilator drugs, vasoconstrictor drugs, anti-tumor drugs, anti-viral drugs, anti-seborrheic drugs, anti-vertigo drugs, deliverance of toxins, deliverance of hormones, delivery of nicotine, compounds for anti-cold, compounds for release of heat, compounds for prevention of contact dermatitis, compounds for prevention of irritants, minerals, amino acids, lipids, herbs and metabolite supplements.

20. The liposome encapsulated composition of claim 12, wherein the bonding solution is a physical bonding solution.

21. The liposome encapsulated composition of claim 12, wherein the bonding solution is a physiochemical bonding solution.

22. The liposome encapsulated bead composition of claim 12, wherein the bonding solution is selected from the group consisting of aragrose, cellulose, sodium alginate, chitosans and polymeric substances.

23. A method of administering at least one active agent to a subject comprising the steps of:
providing the liposome encapsulated composition set forth in claim 12; and
contacting an area of skin or mucous membrane with the liposome encapsulated composition to administer the active agent.

24. The method of claim 23, wherein the at least one active agent is from a class of compounds selected from the group consisting of antifungal drugs, anti-inflammatory drugs, anti-arthritic drugs, corticosteroids, vitamins, whitening agents, nitrous oxide, moisturizers, anabolic drugs, analgesic drugs, anesthetic drugs, anti-asthmatic drugs, antibacterial drugs, antihistaminic drugs, anti-neoplastic drugs, anti-parasitic drugs, vasodilator drugs, vasoconstrictor drugs, anti-tumor drugs, anti-viral drugs, anti-seborrheic drugs, anti-vertigo drugs, deliverance of toxins, deliverance of hormones, delivery of nicotine, compounds for anti-cold, compounds for release of heat, compounds for prevention of contact dermatitis, compounds for prevention of irritants, minerals, amino acids, lipids, herbs and metabolite supplements.
